(19) United States
(12) Patent Application Publication
(10) Pub. No.: US 2021/0023298 A1
(43) Pub. Date: Jan. 28, 2021

(54) METHOD OF REMOVAL OF GAS FROM RESERVOIR

(71) Applicant: BAYER HEALTHCARE LLC, WHIPPANY, NJ (US)

(72) Inventors: MICHAEL MCDERMOTT, PITTSBURGH, PA (US); WILLIAM BARONE, PITTSBURGH, PA (US)

(21) Appl. No.: 17/043,244

(22) PCT Filed: Apr. 18, 2019
(86) PCT No.: PCT/US2019/028139

§ 371 Date: Sep. 29, 2020

Related U.S. Application Data
(60) Provisional application No. 62/659,984, filed on Apr. 19, 2018.
(60) Provisional application No. 62/723,792, filed on Aug. 28, 2018.

(51) Int. Cl. A61M 5/168

(52) U.S. Cl. CPC A61M 5/16822; A61M 2005/1403; A61M 5/16881

(57) ABSTRACT

A fluid injector system includes at least one fluid reservoir having at least one interior surface and defining an internal volume, at least one actuator configured to change the internal volume of the at least one fluid reservoir, and at least one processor. The at least one processor may be programmed or configured to drive the actuator to at least partially fill the at least one fluid reservoir with a fluid from a fluid source, drive the actuator to generate a least a partial vacuum within the internal volume to dislodge one or more gas bubbles adhered to the at least one interior surface and to cause the one or more gas bubbles to coalesce into a coalesced bubble, and drive the actuator to expel the coalesced bubble from an outlet of the at least one fluid reservoir.

### CROSS-REFERENCE TO RELATED APPLICATIONS

This application claims the benefit of U.S. Provisional Patent Application No. 62/659,984, filed on Apr. 19, 2018, and of U.S. Provisional Patent Application No. 62/723,792, filed on Aug. 28, 2018, the disclosures of which are hereby incorporated by reference in their entireties.

### BACKGROUND OF THE DISCLOSURE

### Field of the Disclosure

The present disclosure is directed to fluid delivery applications and, particularly, to a fluid injector system configured to remove gas from at least one fluid reservoir thereof. The present disclosure is further directed to a method of gas removal from at least one fluid reservoir and a computer program product for executing the method.

### Description of the Related Art

In many medical diagnostic and therapeutic procedures, a medical practitioner, such as a physician or radiologist, injects a patient with a fluid. In recent years, a number of injector-actuated syringes and powered injectors for pressurized injection of fluids have been developed for use in procedures such as angiography, computed tomography (CT), and magnetic resonance imaging (MRI). In these procedures, a fluid, such as a contrast agent, may be used to highlight certain internal organs or portions of the body during an imaging process. Meanwhile, saline, or a similar flushing agent, may be used to ensure complete injection of the bolus of the contrast agent.

When drawing a fluid, such as those mentioned above, into a fluid injector system, air or gas bubbles may adhere to the inner surfaces of the system. Under conventional conditions, it can be difficult to remove these air bubbles prior to injection. Typically, the quantities of air are sufficiently small and do not present a concern if injected into the vasculature of the patient. However, there are instances where injection of air, even in low volumes, may be harmful. For example, the injection of air into a vein or artery, for example during an angiography procedure, may cause an air embolism. Even small quantities of air, which may not present the concern of an air embolism, may result in imaging artifacts which may degrade the diagnostic efficacy of an imagining procedure. Further, the presence of air within a fluid injector system may result in transient fluid dynamics, such as a change in flow rate or pressure within the system. These changes within the fluid injector system may lead to further complications, such as inaccurate amounts of fluid being delivered to the patient. The presence of air bubbles within the fluid injector system may also lead the patient or technician to have a negative perception of the injection. Therefore, removing air from a fluid injector system may be advantageous not only to the injection procedure itself, but also to a patient's perception of the procedure.

### SUMMARY OF DISCLOSURE

In view of the disadvantages of injecting air into a patient, there is a need in the art for improved methods of gas removal from fluids in fluid reservoirs of a fluid injector system. The present disclosure is generally directed to systems, methods, and computer program products for removing gas from at least one fluid reservoir of a fluid injector system.

According to various aspects of the present disclosure, a fluid injector system includes at least one actuator configured to change the internal volume of the at least one fluid reservoir, at least one fluid reservoir having at least one interior surface and defining an internal volume, and at least one processor. The at least one processor is programmed or configured to drive the actuator to at least partially fill the at least one fluid reservoir with a fluid from a fluid source, drive the actuator to generate an at least a partial vacuum within the internal volume to dislodge one or more gas bubbles adhered to the at least one interior surface and to cause the one or more gas bubbles to coalesce into a coalesced bubble, and drive the actuator to expel the coalesced bubble from an outlet of the at least one fluid reservoir. According to some aspects of the present disclosure, the at least one processor is further programmed or configured to, prior to driving the actuator to generate at least the partial vacuum within the internal volume, close the outlet of the at least one fluid reservoir to fluidly isolate the internal volume. According to certain aspects, the at least one processor is further programmed or configured to, after closing the outlet of the at least one fluid reservoir, drive the actuator to pressurize the coalesced bubble. According to some aspects, the at least one processor is further programmed or configured to, prior to driving the actuator to expel the coalesced bubble from an outlet of the at least one fluid reservoir, open the outlet of the at least one fluid reservoir to provide fluid communication between the internal volume and one of the fluid source and a fluid outlet pathway. According to some aspects of the present disclosure, the at least one processor is further programmed or configured to vibrate, oscillate, or provide an impact force on at least one of the at least one interior surfaces to dislodge the one or more gas bubbles.

According to some aspects of the present disclosure, the at least one fluid reservoir includes a syringe. The actuator may include a piston configured to move reciprocally to change the internal volume of the syringe. The at least one interior surface may further include a surface of the plunger. According to some aspects, the syringe includes a rolling diaphragm syringe. The at least one interior surface includes an inner surface of the rolling diaphragm syringe. The piston is releasably connected to a proximal end wall of the syringe and is configured to reciprocally move the proximal end wall of the rolling diaphragm syringe.

According to some aspects of the present disclosure, driving the actuator to at least partially fill the at least one fluid reservoir includes moving a piston of the fluid injector system operatively connected to the at least one fluid reservoir in a first direction. Driving the actuator to generate the at least partial vacuum within the internal volume includes moving the piston in the first direction. Driving the actuator to expel the coalesced bubble includes moving the piston in a second direction opposite the first direction.

According to some aspects of the present disclosure, the at least one fluid reservoir may further include a valve in fluid communication with the outlet of the at least one fluid reservoir. The valve has at least a first open position and a second closed position. Closing the outlet of the at least one fluid reservoir includes moving the valve to the second closed position. According to some aspects, there is fluid communication between the internal volume of the at least one fluid reservoir and the fluid source when the valve is in the first open position. The valve may further include a third open position allowing fluid communication between the internal volume of the at least one fluid reservoir and a fluid outlet pathway.

According to some aspects of the disclosure, the at least partial vacuum generated within the internal volume is sufficient to extract at least a portion of a dissolved gas from the fluid. The dissolved gas that is extracted coalesces into the coalesced bubble.

According to some aspects of the present disclosure, the at least one processor is further programmed or configured to drive the actuator to prime a fluid path set in fluid communication with a fluid outlet pathway after the coalesced bubble is expelled from the at least one fluid reservoir. According to some aspects, the at least one processor is further programmed or configured to determine the amount of vacuum necessary to dislodge the one or more gas bubbles from the at least one interior surface based on at least one of a surface tension of the fluid, a surface tension of the gas, a surface texture of the at least one interior surface, interfacial surface energy between the at least one interior surface and the gas, a weight of the fluid above the one or more gas bubbles in the fluid reservoir, and a buoyancy of the one or more gas bubble in the fluid. According to some aspects, the at least one processor is further programmed or configured to measure the pressure within the internal volume of the fluid reservoir and adjust the at least partial vacuum within the internal volume based on the measured pressure. According to some aspects, adjusting the at least partial vacuum includes at least one of increasing or decreasing a speed of retraction of the actuator and increasing or reducing a diameter of at least a portion of a fluid path set in fluid communication with the internal volume of the fluid reservoir.

According to some aspects of the present disclosure, a method for removing gas bubbles from at least one fluid reservoir of a fluid injector system includes driving an actuator to at least partially fill the at least one fluid reservoir with a fluid from a fluid source, driving the actuator to generate an at least partial vacuum within an internal volume of the at least one fluid reservoir to dislodge one or more gas bubbles adhered to least one interior surface of the at least one fluid reservoir and to cause the one or more gas bubbles to coalesce into a coalesced bubble, and driving the actuator to expel the coalesced bubble from an outlet of the at least one fluid reservoir.

According to some aspects, the method may further include, prior to driving the actuator to generate at least the partial vacuum within the internal volume, closing the outlet of the at least one fluid reservoir to fluidly isolate the internal volume. According to some aspects, the method may further include, after closing the outlet of the at least one fluid reservoir, driving the actuator to pressurize the coalesced bubble.

According to some aspects, the method may further include, prior to driving the actuator to expel the coalesced bubble from an outlet of the at least one fluid reservoir, opening the outlet of the at least one fluid reservoir to provide fluid communication between the internal volume and one of the fluid source and a fluid outlet pathway.

According to some aspects of the present disclosure, driving the actuator to at least partially fill the at least one fluid reservoir includes moving a piston of the fluid injector system operatively connected to the at least one fluid reservoir in a first direction. Driving the actuator to generate the at least partial vacuum within the internal volume includes moving the piston in the first direction. Driving the actuator to expel the coalesced bubble includes moving the piston in a second direction opposite the first direction.

According to some aspects, the method may further include vibrating, oscillating, or providing an impact force on at least one of the at least one interior surfaces to dislodge the one or more gas bubbles.

According to some aspects of the present disclosure, closing the outlet of the at least one fluid reservoir includes moving a valve at the outlet from a first open position where the internal volume is in fluid communication with the fluid source to a second closed position where the internal volume is fluidly isolated from the fluid source. According to some aspects, there is fluid communication between the internal volume of the at least one fluid reservoir and the fluid source when the valve is in the first open position. The valve may further include a third open position allowing fluid communication between the internal volume of the at least one fluid reservoir and a fluid outlet pathway. According to some aspects, the method may further include driving the actuator to prime a fluid path set in fluid communication with reservoir fluid outlet pathway after the coalesced bubble is expelled from the at least one fluid reservoir.

According to some aspects of the present disclosure, the method may further include determining the amount of vacuum necessary to dislodge the one or more gas bubbles from the at least one interior surface based one at least one of a surface tension of the fluid, a surface tension of the gas, a surface texture of the at least one interior surface, and buoyancy of the one or more gas bubble in the fluid. According to some aspects, the method may further include measuring the pressure within the internal volume of the fluid reservoir and adjusting the at least partial vacuum within the internal volume based on the measured pressure. According to some aspects of the present disclosure, adjusting the at least partial vacuum includes at least one of: increasing or decreasing a speed of retraction of the actuator; and increasing or reducing a diameter of at least a portion of a fluid path set in fluid communication with the internal volume of the fluid reservoir.

According to some aspects of the present disclosure, a method for removing gas bubbles from at least one fluid filled fluid reservoir of a fluid injector system includes generating at least a partial vacuum within an internal volume of the at least one fluid reservoir dislodging one or more gas bubbles adhered to least one interior surface of the at least one fluid reservoir. The vacuum causes the one or more dislodged gas bubbles to enlarge and coalesce into a coalesced bubble. The method may further include expelling the coalesced bubble from an outlet of the at least one fluid reservoir. According to some aspects of the present disclosure, the method may further include closing the outlet of the at least one fluid reservoir prior to generating the at least partial vacuum within the internal volume.

According to some aspects, the method may further include pressurizing the coalesced bubble after closing the outlet of the at least one fluid reservoir. According to some aspects of the present disclosure, the method may further include opening the outlet of the at least one fluid reservoir prior to expelling the coalesced bubble from an outlet of the at least one fluid reservoir.

According to some aspects of the present disclosure, the at least one fluid reservoir may comprise a syringe. Generating the at least partial vacuum within an internal volume includes driving a piston of the fluid injection system in a first direction to increase the internal volume. Expelling the coalesced bubble including driving the piston of the fluid injection system in a second direction to decrease the internal volume.

According to some aspects of the present disclosure, the method may further include priming a fluid path set in fluid communication with the at least one fluid reservoir after the coalesced bubble is expelled from the at least one fluid reservoir.

According to some aspects of the present disclosure, generating a vacuum in the internal volume includes generating the at least partial vacuum sufficient to extract at least a portion of a dissolved gases from the fluid, and wherein the dissolved gas that is extracted coalesces into the coalesced bubble.

According to some aspects of the present disclosure, a computer program product includes at least one non-transitory computer-readable medium including program instructions for removing gas from at least one fluid reservoir of a fluid injector system. When executed by at least one processor, the program instructions cause the at least one processor to drive at least one actuator of the fluid injector system to at least partially fill the at least one fluid reservoir with a fluid from a fluid source, drive the at least one actuator to generate an at least partial vacuum within an internal volume of the at least one fluid reservoir to dislodge one or more gas bubbles adhered to an at least one interior surface of the at least one fluid reservoir and to cause the one or more gas bubbles to coalesce into a coalesced bubble, and drive the actuator to expel the coalesced bubble from an outlet of the at least one fluid reservoir. According to some aspects of the present disclosure, the instructions further cause the at least one processor to, prior to driving the actuator to generate the at least partial vacuum within the internal volume, close the outlet of the at least one fluid reservoir to fluidly isolate the internal volume. According to some aspects of the present disclosure, the instructions further cause the at least one processor to, after closing the outlet of the at least one fluid reservoir, drive the actuator to pressurize the coalesced bubble. According to some aspects of the present disclosure, the instructions further cause the at least one processor to vibrate, oscillate, or provide an impact force on at least one of the at least one interior surfaces to dislodge the one or more gas bubbles.

According to some aspects of the present disclosure, the instructions further cause the at least one processor to, prior to driving the actuator to expel the coalesced bubble from an outlet of the at least one fluid reservoir, open the outlet of the at least one fluid reservoir to provide fluid communication between the internal volume and one of the fluid source and a fluid outlet pathway.

According to some aspects of the present disclosure, the syringe includes a rolling diaphragm syringe. The at least one interior surface includes an inner surface of the rolling diaphragm syringe. The piston is releasably connected to a proximal end wall of the rolling diaphragm syringe and is configured to reciprocally move the proximal end wall of the rolling diaphragm syringe.

According to some aspects of the present disclosure, driving the actuator to at least partially fill the at least one fluid reservoir includes moving a piston of the fluid injector system operatively connected to the at least one fluid reservoir in a first direction. Driving the actuator to generate the at least partial vacuum within the internal volume includes moving the piston in the first direction. Driving the actuator to expel the coalesced bubble includes moving the piston in a second direction opposite the first direction.

According to some aspects of the present disclosure, the at least one fluid reservoir may further include a valve in fluid communication with the outlet of the at least one fluid reservoir. The valve has at least a first open position and a second closed position. Closing the outlet of the at least one fluid reservoir includes moving the valve to the second closed position. According to some aspects of the present disclosure, there is fluid communication between the internal volume of the at least one fluid reservoir and the fluid source when the valve is in the first open position. The valve may further include a third open position allowing fluid communication between the internal volume of the at least one fluid reservoir and a fluid outlet pathway.

According to some aspects of the present disclosure, the at least partial vacuum generated within the internal volume is sufficient to extract at least a portion of a dissolved gas from the fluid. The dissolved gas that is extracted coalesces into the coalesced bubble. According to some aspects of the present disclosure, the instructions further cause the at least one processor to drive the actuator to prime a fluid path set in fluid communication with a fluid outlet pathway after the coalesced bubble is expelled from the fluid reservoir.

According to some aspects of the present disclosure, the instructions further cause the at least one processor to determine the amount of vacuum necessary to dislodge the one or more gas bubbles from the at least one interior surface based on at least one of a surface tension of the fluid, a surface tension of the gas, a surface texture of the at least one interior surface, interfacial surface energy between the at least one interior surface and the gas, a weight of the fluid above the one or more gas bubbles in the fluid reservoir, and a buoyancy of the one or more gas bubbles in the fluid. According to some aspects of the present disclosure, the instructions further cause the at least one processor to measure the pressure within the internal volume of the fluid reservoir and adjust the at least partial vacuum within the internal volume based on the measured pressure.

According to some aspects of the present disclosure, adjusting the at least partial vacuum includes at least one of increasing or decreasing a speed of retraction of the actuator and increasing or reducing a diameter of at least a portion of a fluid path set in fluid communication with the internal volume of the fluid reservoir.

Various other aspects of the system, computer program product, and method for removing gas from at least one fluid reservoir are disclosed in one or more of the following numbered clauses:

Clause 1. A fluid injector system, comprising: at least one actuator configured to change the internal volume of the at least one fluid reservoir; at least one fluid reservoir having at least one interior surface and defining an internal volume; and at least one processor programmed or configured to: drive the actuator to at least partially fill the at least one fluid reservoir with a fluid from a fluid source; drive the actuator to generate an at least a partial vacuum within the internal volume to dislodge one or more gas bubbles adhered to the at least one interior surface and to cause the one or more gas bubbles to coalesce into a coalesced bubble; and drive the actuator to expel the coalesced bubble from an outlet of the at least one fluid reservoir.

Clause 2. The fluid injector system of clause 1, wherein the at least one processor is further programmed or configured to, prior to driving the actuator to generate at least the partial vacuum within the internal volume, close the outlet of the at least one fluid reservoir to fluidly isolate the internal volume.

Clause 3. The fluid injector system of clause 2, wherein the at least one processor is further programmed or configured to, after closing the outlet of the at least one fluid reservoir, drive the actuator to pressurize the coalesced bubble.

Clause 4. The fluid injector system of clause 2 or 3, wherein the at least one processor is further programmed or configured to, prior to driving the actuator to expel the coalesced bubble from an outlet of the at least one fluid reservoir, open the outlet of the at least one fluid reservoir to provide fluid communication between the internal volume and one of the fluid source and a fluid outlet pathway.

Clause 5. The fluid injector system of any of clauses 1 to 4, wherein the at least one fluid reservoir comprises a syringe, and wherein the actuator comprises a piston configured to move reciprocally to change the internal volume of the syringe.

Clause 6. The fluid injector system of clause 5, wherein the syringe further comprises a plunger slideable within the syringe, wherein the at least one interior surface further comprises a surface of the plunger, and wherein the piston is releasably connected to the plunger and is configured to reciprocally move the plunger within the syringe.

Clause 7. The fluid injector system of clause 5, wherein the syringe comprises a rolling diaphragm syringe, wherein the at least one interior surface comprises an inner surface of the syringe, and wherein the piston is releasably connected to a proximal end wall of the syringe and is configured to reciprocally move the proximal end wall of the syringe.

Clause 8. The fluid injector system of any of clauses 1 to 7, wherein driving the actuator to at least partially fill the at least one fluid reservoir comprises moving a piston of the fluid injector system operatively connected to the at least one fluid reservoir in a first direction, wherein driving the actuator to generate the at least partial vacuum within the internal volume comprises moving the piston in the first direction, and wherein driving the actuator to expel the coalesced bubble comprises moving the piston in a second direction opposite the first direction.

Clause 9. The fluid injector system of any of clauses 1 to 8, wherein the at least one processor is further programmed or configured to vibrate or oscillate at least one of the at least one interior surfaces to dislodge the one or more gas bubbles.

Clause 10. The fluid injector system of any of clauses 2 to 9, wherein the at least one fluid reservoir further comprises a valve in fluid communication with the outlet of the at least one fluid reservoir, wherein the valve has at least a first open position and a second closed position wherein closing the outlet of the at least one fluid reservoir comprises moving the valve to the second closed position.

Clause 11. The fluid injector system of clause 10, wherein there is fluid communication between the internal volume of the at least one fluid reservoir and the fluid source when the valve is in the first open position, and wherein the valve further comprises a third open position allowing fluid communication between the internal volume of the at least one fluid reservoir and a fluid outlet pathway.

Clause 12. The fluid injector system of clause 10 or 11, wherein the valve comprises a stopcock.

Clause 13. The fluid injector system of any of clauses 1 to 12, wherein the at least partial vacuum generated within the internal volume is sufficient to extract at least a portion of a dissolved gas from the fluid, and wherein the dissolved gas that is extracted coalesces into the coalesced bubble.

Clause 14. The fluid injector system of any of clauses 1 to 13, wherein the at least one processor is further programmed or configured to drive the actuator to prime a fluid path set in fluid communication with a fluid outlet pathway after the coalesced bubble is expelled from the at least one fluid reservoir.

Clause 15. The fluid injector system of any of clauses 1 to 14, wherein the at least one processor is further programmed or configured to determine the amount of vacuum necessary to dislodge the one or more gas bubbles from the at least one interior surface based on at least one of a surface tension of the fluid, a surface tension of the gas, a surface texture of the at least one interior surface, interfacial surface energy between the at least one interior surface and the gas, a weight of the fluid above the one or more gas bubbles in the fluid reservoir, and a buoyancy of the one or more gas bubble in the fluid.

Clause 16. The fluid injector system of any of clauses 1 to 15, wherein the at least one processor is further programmed or configured to: measure the pressure within the internal volume of the fluid reservoir; and adjust the at least partial vacuum within the internal volume based on the measured pressure.

Clause 17. The fluid injector system of clause 16, wherein adjusting the at least partial vacuum comprises at least one of: increasing or decreasing a speed of retraction of the actuator; and increasing or reducing a diameter of at least a portion of a fluid path set in fluid communication with the internal volume of the fluid reservoir.

Clause 18. A method for removing gas bubbles from at least one fluid reservoir of a fluid injector system, the method comprising: driving an actuator to at least partially fill the at least one fluid reservoir with a fluid from a fluid source; driving the actuator to generate an at least partial vacuum within an internal volume of the at least one fluid reservoir to dislodge one or more gas bubbles adhered to least one interior surface of the at least one fluid reservoir and to cause the one or more gas bubbles to coalesce into a coalesced bubble; and driving the actuator to expel the coalesced bubble from an outlet of the at least one fluid reservoir.

Clause 19. The method of clause 18, further comprising: prior to driving the actuator to generate at least the partial vacuum within the internal volume, closing the outlet of the at least one fluid reservoir to fluidly isolate the internal volume.

Clause 20. The method of clause 19, further comprising: after closing the outlet of the at least one fluid reservoir, driving the actuator to pressurize the coalesced bubble.

Clause 21. The method of clause 19 or 20, further comprising: prior to driving the actuator to expel the coalesced bubble from an outlet of the at least one fluid reservoir, opening the outlet of the at least one fluid reservoir to provide fluid communication between the internal volume and one of the fluid source and a fluid outlet pathway.

Clause 22. The method of any of clauses 18 to 21, wherein the at least one fluid reservoir comprises a syringe, wherein the actuator comprises a piston, and wherein driving the actuator comprises linearly moving the piston to change the internal volume of the syringe.

Clause 23. The method of any of clauses 18 to 22, wherein driving the actuator to at least partially fill the at least one fluid reservoir comprises moving a piston of the fluid injector system operatively connected to the at least one fluid reservoir in a first direction, wherein driving the actuator to generate the at least partial vacuum within the internal volume comprises moving the piston in the first direction, and wherein driving the actuator to expel the coalesced bubble comprises moving the piston in a second direction opposite the first direction.

Clause 24. The method of any of clauses 18 to 23, further comprising vibrating at least one of the at least one interior surfaces to dislodge the one or more gas bubbles.

Clause 25. The method of any of clauses 18 to 24, wherein closing the outlet of the at least one fluid reservoir comprises moving a valve at the outlet from a first open position where the internal volume is in fluid communication with the fluid source to a second closed position where the internal volume is fluidly isolated from the fluid source.

Clause 26. The method of clause 25, wherein there is fluid communication between the internal volume of the at least one fluid reservoir and the fluid source when the valve is in the first open position, and wherein the valve further comprises a third open position allowing fluid communication between the internal volume of the at least one fluid reservoir and a fluid outlet pathway.

Clause 27. The method of any of clauses 18 to 26, further comprising: driving the actuator to prime a fluid path set in fluid communication with reservoir fluid outlet pathway after the coalesced bubble is expelled from the at least one fluid reservoir.

Clause 28. The method of any of clauses 18 to 27, further comprising: determining the amount of vacuum necessary to dislodge the one or more gas bubbles from the at least one interior surface based one at least one of a surface tension of the fluid, a surface tension of the gas, a surface texture of the at least one interior surface, and buoyancy of the one or more gas bubble in the fluid.

Clause 29. The method of any of clauses 18 to 28, further comprising: measuring the pressure within the internal volume of the fluid reservoir; and adjusting the at least partial vacuum within the internal volume based on the measured pressure.

Clause 30. The method of clause 29, wherein adjusting the at least partial vacuum comprises at least one of: increasing or decreasing a speed of retraction of the actuator; and increasing or reducing a diameter of at least a portion of a fluid path set in fluid communication with the internal volume of the fluid reservoir.

Clause 31. A method for removing gas bubbles from at least one fluid filled fluid reservoir of a fluid injector system, the method comprising: generating at least a partial vacuum within an internal volume of the at least one fluid reservoir dislodging one or more gas bubbles adhered to least one interior surface of the at least one fluid reservoir, wherein the vacuum causes the one or more dislodged gas bubbles to enlarge and coalesce into a coalesced bubble; and expelling the coalesced bubble from an outlet of the at least one fluid reservoir.

Clause 32. The method of clause 31, further comprising: closing the outlet of the at least one fluid reservoir prior to generating the at least partial vacuum within the internal volume.

Clause 33. The method of clause 32, further comprising: pressurizing the coalesced bubble after closing the outlet of the at least one fluid reservoir.

Clause 34. The method of clause 31 or 33, further comprising: opening the outlet of the at least one fluid reservoir prior to expelling the coalesced bubble from an outlet of the at least one fluid reservoir.

Clause 35. The method of any of clauses 31 to 34, wherein the at least one fluid reservoir comprises a syringe, wherein generating the at least partial vacuum within an internal volume comprises driving a piston of the fluid injection system in a first direction to increase the internal volume, and wherein expelling the coalesced bubble comprising driving the piston of the fluid injection system in a second direction to decrease the internal volume.

Clause 36. The method of any of clauses 31 to 35, wherein dislodging the one or more gas bubbles comprises vibrating at least a portion of an interior surface of the at least one fluid reservoir.

Clause 37. The method of clause 35 or 36, wherein dislodging the one or more gas bubbles comprises reciprocally vibrating the piston.

Clause 38. The method of any of clauses 31 to 37, further comprising: priming a fluid path set in fluid communication with the at least one fluid reservoir after the coalesced bubble is expelled from the at least one fluid reservoir.

Clause 39. The method of any of clauses 31 to 38, wherein generating a vacuum in the internal volume comprises generating the at least partial vacuum sufficient to extract at least a portion of a dissolved gases from the fluid, and wherein the dissolved gas that is extracted coalesces into the coalesced bubble.

Clause 40. A computer program product comprising at least one non-transitory computer-readable medium including program instructions for removing gas from at least one fluid reservoir of a fluid injector system, that, when executed by at least one processor, cause the at least one processor to: drive at least one actuator of the fluid injector system to at least partially fill the at least one fluid reservoir with a fluid from a fluid source; drive the at least one actuator to generate an at least partial vacuum within an internal volume of the at least one fluid reservoir to dislodge one or more gas bubbles adhered to an at least one interior surface of the at least one fluid reservoir and to cause the one or more gas bubbles to coalesce into a coalesced bubble; and drive the actuator to expel the coalesced bubble from an outlet of the at least one fluid reservoir.

Clause 41. The computer program product of clause 40, wherein the instructions further cause the at least one processor to, prior to driving the actuator to generate the at least partial vacuum within the internal volume, close the outlet of the at least one fluid reservoir to fluidly isolate the internal volume.

Clause 42. The computer program product of clause 40 or 41, wherein the instructions further cause the at least one processor to, after closing the outlet of the at least one fluid reservoir, drive the actuator to pressurize the coalesced bubble.

Clause 43. The computer program product of clause 41 or 42, wherein the instructions further cause the at least one processor to, prior to driving the actuator to expel the coalesced bubble from an outlet of the at least one fluid reservoir, open the outlet of the at least one fluid reservoir to provide fluid communication between the internal volume and one of the fluid source and a fluid outlet pathway.

Clause 44. The computer program product of any of clauses 40 to 43, wherein the at least one fluid reservoir comprises a syringe, and wherein the actuator comprises a piston configured to move reciprocally to change the internal volume of the syringe.

Clause 45. The computer program product of clause 44, wherein the syringe further comprises a plunger slideable within the syringe, wherein the at least one interior surface further comprises a surface of the plunger, and wherein the piston is releasably connected to the plunger and is configured to reciprocally move the plunger within the syringe.

Clause 46. The computer program product of clause 44, wherein the syringe comprises a rolling diaphragm syringe, wherein the at least one interior surface comprises an inner surface of the syringe, and wherein the piston is releasably connected to a proximal end wall of the syringe and is configured to reciprocally move the proximal end wall of the syringe.

Clause 47. The computer program product of any of clauses 40 to 46, wherein driving the actuator to at least partially fill the at least one fluid reservoir comprises moving a piston of the fluid injector system operatively connected to the at least one fluid reservoir in a first direction, wherein driving the actuator to generate the at least partial vacuum within the internal volume comprises moving the piston in the first direction, and wherein driving the actuator to expel the coalesced bubble comprises moving the piston in a second direction opposite the first direction.

Clause 48. The computer program product of any of clauses 40 to 47, wherein the instructions further cause the at least one processor to vibrate or oscillate at least one of the at least one interior surfaces to dislodge the one or more gas bubbles.

Clause 49. The computer program product of any of clauses 41 to 48, wherein the at least one fluid reservoir further comprises a valve in fluid communication with the outlet of the at least one fluid reservoir, wherein the valve has at least a first open position and a second closed position wherein closing the outlet of the at least one fluid reservoir comprises moving the valve to the second closed position.

Clause 50. The computer program product of clause 49, wherein there is fluid communication between the internal volume of the at least one fluid reservoir and the fluid source when the valve is in the first open position, and wherein the valve further comprises a third open position allowing fluid communication between the internal volume of the at least one fluid reservoir and a fluid outlet pathway.

Clause 51. The computer program product of clause 49 or 50, wherein the valve comprises a stopcock.

Clause 52. The computer program product of any of clauses 40 to 51, wherein the at least partial vacuum generated within the internal volume is sufficient to extract at least a portion of a dissolved gas from the fluid, and wherein the dissolved gas that is extracted coalesces into the coalesced bubble.

Clause 53. The computer program product of any of clauses 40 to 52, wherein the instructions further cause the at least one processor to drive the actuator to prime a fluid path set in fluid communication with a fluid outlet pathway after the coalesced bubble is expelled from the at least one fluid reservoir.

Clause 54. The computer program product of any of clauses 40 to 53, wherein the instructions further cause the at least one processor to determine the amount of vacuum necessary to dislodge the one or more gas bubbles from the at least one interior surface based on at least one of a surface tension of the fluid, a surface tension of the gas, a surface texture of the at least one interior surface, interfacial surface energy between the at least one interior surface and the gas, a weight of the fluid above the one or more gas bubbles in the fluid reservoir, and a buoyancy of the one or more gas bubbles in the fluid.

Clause 55. The computer program product of any of clauses 40 to 54, wherein the instructions further cause the at least one processor to: measure the pressure within the internal volume of the fluid reservoir; and adjust the at least partial vacuum within the internal volume based on the measured pressure.

Clause 56. The computer program product of clause 55, wherein adjusting the at least partial vacuum comprises at least one of: increasing or decreasing a speed of retraction of the actuator; and increasing or reducing a diameter of at least a portion of a fluid path set in fluid communication with the internal volume of the fluid reservoir.

These and other features and characteristics of fluid injector systems, as well as computer program products and methods of operation and functions of the related elements of structures and the combination of parts and economies of manufacture, will become more apparent upon consideration of the following description and the appended claims with reference to the accompanying drawings, all of which form a part of this specification. It is to be expressly understood, however, that the drawings are for the purpose of illustration and description only.

### BRIEF DESCRIPTION OF THE DRAWINGS

FIG. 1 is a perspective view of a multi-fluid delivery system, according to one example of the present disclosure;

FIG. 2 is a perspective view of the multi-patient disposable set (MUDS) within the multi-fluid delivery system of FIG. 1;

FIG. 3A is a perspective view of a connection interface prior to connecting a single-use disposable set (SUDS) connector with a multi-fluid delivery system;

FIG. 3B is a perspective view of the connection interface of FIG. 3A showing the SUDS connector connected with the multi-fluid delivery system;

FIG. 4 is a schematic view of an electronic control system of a multi-fluid delivery system according to examples herein;

FIG. 5 is a perspective view of a multi-fluid delivery system, according to examples of the present disclosure;

FIG. 6A is a side cross-sectional view of a syringe according to examples of the present disclosure with the syringe shown in an unrolled state;

FIG. 6B is a side cross-sectional view of the syringe of FIG. 6A with the syringe shown in a rolled state;

FIG. 7 is a flowchart of a method for gas removal from a closed fluid reservoir according to examples of the present disclosure;

FIG. 8A-8B are schematic illustrations of an at least partial vacuum being placed on a closed fluid reservoir according to examples of the present disclosure;

FIG. 8C is a schematic illustration of the at least partial vacuum being placed on a closed fluid reservoir of FIGS. 8A-8B, with a coalesced gas bubble formed therein;

FIG. 9 is a graphical representation of the pressure in a fluid reservoir versus time in an example of the present disclosure;

FIGS. 10A-10B are illustrations of open fluid reservoirs in examples of the present disclosure;

FIG. 11 is a flowchart of a method for gas removal from an open fluid reservoir in an example of the present disclosure;

FIGS. 12A-12B are illustrations of an at least partial vacuum being placed on an open fluid reservoir by varying piston retraction speed, in an example of the present disclosure;

FIGS. 13A-13B are illustrations of an at least partial vacuum being placed on an open fluid reservoir by altering fluid path diameter, in an example of the present disclosure;

FIGS. 14A-14B are illustrations of fluid reservoirs having a gas collection chamber in examples of the present disclosure;

FIG. 15 is an illustration of a fluid reservoir having a gas collection chamber in an example of the present disclosure;

FIG. 16 is a graphical representation of bubble size versus vacuum pressure necessary to dislodge the bubbles in an example of the present disclosure; and

FIG. 17 is a graphical representation of retraction of the piston versus vacuum pressure necessary to dislodge the bubbles in an example of the present disclosure.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to a syringe of a multi-patient disposable set, the term “proximal” refers to a portion of a syringe nearest a piston for delivering fluid from a syringe. Spatial or directional terms, such as “left”, “right”, “inner”, “outer”, “above”, “below”, and the like, are not to be considered as limiting as the various features can assume various alternative orientations. All numbers used in the specification and claims are to be understood as being modified in all instances by the term “about”. The term “about” means a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

As used herein, the term “at least a partial vacuum” means a reduction of the pressure inside the fluid reservoir relative to the pressure outside the fluid reservoir. For example, if the outside of the fluid reservoir is at atmospheric pressure and the interior of the fluid reservoir is depressurized to at least a partial vacuum, the interior of the fluid reservoir may be at a reduced pressure of at least 0.1 atm less than the exterior of the fluid reservoir, in other embodiments at least 0.25 atm less than the exterior pressure of the fluid reservoir, and in other embodiments at a reduced pressure of at least 0.5 atm less than the exterior of the fluid reservoir. As used herein, specific values for pressure refer to gauge pressure unless otherwise noted. For example, a pressure of 0 atm or 0 psi corresponds to standard atmospheric pressure (i.e. 1 atm or 14.7 psi absolute pressure). As used herein, the term “air” is used synonymously with “gas” and can mean any gas bubble or any gas dissolved in fluid.

It is also to be understood that the specific devices and processes illustrated in the attached drawings, and described in the following specification, are simply exemplary examples of the disclosure. Hence, specific dimensions and other physical characteristics related to the examples disclosed herein are not to be considered as limiting.

When used in relation to a fluid reservoir, such as a syringe, a rolling diaphragm or single-use disposable set connector, the term “distal” refers to a portion of the fluid reservoir nearest to a patient. When used in relation to a fluid reservoir, such as a syringe, a rolling diaphragm or single-use disposable set connector, the term “proximal” refers to a portion of the fluid reservoir nearest to the injector system.

The term “open”, when used to refer to a fluid delivery component, means that the system is in fluid connection with an outlet to atmospheric pressure, for example through a nozzle or the open end of a tubing component or catheter. In an open system, fluid flow may be constrained or restricted, for example by forcing a fluid through a small diameter fluid path where flow may be determined by physical parameters of the system and the fluid, such as tubing diameter, fluid path constrictions, applied pressure, viscosity, etc. The term “closed” or “closeable”, when used to refer to a fluid delivery component, means that the system has at least one state in which the component is not in fluid connection with an outlet under atmospheric pressure or the fluid in the fluid reservoir is fluidly isolated, for example where fluid flow is stopped by a valve, such as a stopcock, high crack pressure valve, pinch valve, and the like, that closes a fluid pathway. As used herein, the phrase “at least partial vacuum” means a gauge pressure less than the current atmospheric pressure, for example from −14.7 psi to −0.1 psi.

Referring to the drawings in which like reference characters refer to like parts throughout the several views thereof, one embodiment of the present disclosure is generally directed to a multi-fluid medical injector/injector system 100 (hereinafter “fluid injector system 100”) having a multi-patient disposable set (MUDS) 130 configured for delivering fluid to a patient using a single-use disposable set (SUDS) 190 connector. The fluid injector system 100 includes multiple components as individually described herein. Generally, the fluid injector system 100 has a powered injector or other administration device and a fluid delivery set intended to be associated with the injector to deliver one or more fluids from one or more multi-dose containers under pressure into a patient, as described herein. The various devices, components, and features of the fluid injector system 100 and the fluid delivery set associated therewith are likewise described in detail herein. While the various embodiments of the methods and processor are shown with reference to an injector system having a MUDS and SUDS configuration, the disclosure is not limited to such an injector system and may be utilized in other syringe based injector systems, such as but not limited to those described in U.S. Pat. Nos. 7,553,294, 7,563,249, 8,945,051, 9,173,995, 10,124,110; and U.S. application Ser. Nos. 15/305,285, 15/541,573, 15/568,505; the disclosures of each of which are incorporated herein in their entirety by this reference.

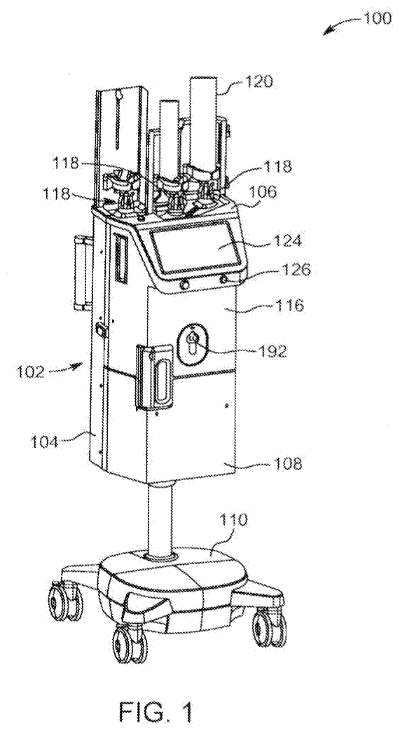

With reference to FIG. 1, a fluid injector system 100 according to one embodiment includes an injector housing 102 having opposed lateral sides 104, a distal or upper end 106, and a proximal or lower end 108. The housing 102 encloses the various mechanical drive components, electrical and power components necessary to drive the mechanical drive components, and control components, such as electronic memory and electronic control devices (hereinafter electronic control device(s)), used to control operation of reciprocally movable pistons 103 associated with the fluid injector system 100 described herein. Such pistons 103 may be reciprocally operable via electro-mechanical drive components such as a ball screw shaft driven by a motor, a voice coil actuator, a rack-and-pinion gear drive, a linear motor, and the like. In some examples, at least some of the mechanical drive components, electrical and power components, and control components may be provided on the base 110.

With continued reference to FIG. 1, the fluid injector system 100 may have at least one door 116 that encloses at least some of the MUDS, the mechanical drive components, electrical and power components, and control components.

The fluid injector system 100 may include at least one bulk fluid connector 118 for connection with at least one bulk fluid source 120. In some examples, a plurality of bulk fluid connectors 118 may be provided. For example, as shown in the fluid injector embodiment illustrated in FIG. 1, three bulk fluid connectors 118 may be provided in a side-by-side or other arrangement. In some examples, the at least one bulk fluid connector 118 may be a spike configured for removably connecting to the at least one bulk fluid source 120, such as a vial, a bottle, or a bag. The at least one bulk fluid connector 118 may be formed on the multi-patient disposable set, as described herein. The at least one bulk fluid source 120 may be configured for receiving a medical fluid, such as saline, contrast solution, or other medical fluid, for delivery to the fluid injector system 100.

With continued reference to FIG. 1, the fluid injector system 100 includes one or more user interfaces 124, such as a graphical user interface (GUI) display window. The user interface 124 may display information pertinent to a fluid injection procedure involving fluid injector system 100, such as injection status or progress, current flow rate, fluid pressure, and volume remaining in the at least one bulk fluid source 120 connected to the fluid injector system 100 and may be a touch screen GUI that allows an operator to input commands and/or data for operation of fluid injector system 100. While the user interface 124 is shown on the injector housing 102, such user interface 124 may also be in the form of, or the fluid injector system 100 may additionally have, a remote display that is wired or wirelessly linked to the housing 102 and control and mechanical elements of fluid injector system 100, for example in a remote room designed to shield the user from exposure to x-rays. In some examples, the user interface 124 may be a tablet computer that is detachably connected to the housing 102 and is in wired or wirelessly linked communication with the housing 102. Additionally, the fluid injector system 100 and/or user interface 124 may include at least one control button 126 for tactile operation by an attendant operator of the fluid injector system 100. In certain examples, the at least one control button 126 may be part of a keyboard for inputting commands and/or data by the operator. The at least one control button 126 may be hard-wired to the electronic control device(s) associated with the fluid injector system 100 to provide direct input to the electronic control device(s). The at least one control button 126 may also be a graphical part of the user interface 124, such as a touch screen. In either arrangement, the at least one control button 126 desirably provides certain individual control features to the attendant operator of the fluid injector system 100, such as, but not limited to: (1) acknowledging that a multi-patient disposable set has been loaded or unloaded; (2) selecting or programing an injection protocol; (3) filling/purging of the fluid injector system 100; (4) inputting information and/or data related to the patient and/or injection procedure; (5) preloading the fluid injector system 100; and (6) initiating/stopping an injection procedure. The user interface 124 and/or any electronic processing units associated with the fluid injector system 100 may be wired or wirelessly connected to an operation and/or data storage system such as a hospital network system.

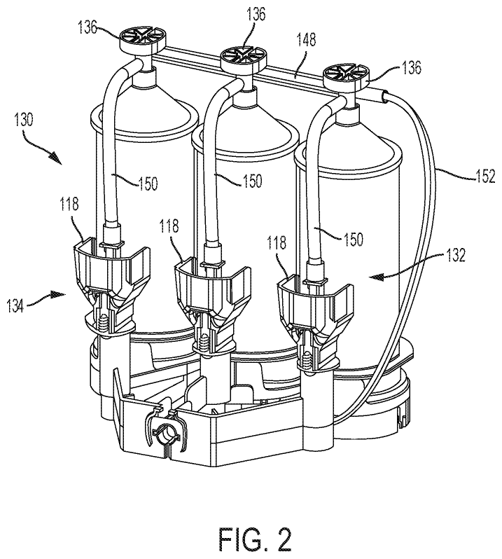

With reference to FIG. 2, the fluid injector system 100 includes a MUDS 130 that is removably connected to the fluid injector system 100 for delivering one or more fluids from the one or more bulk fluid sources 120 to the patient. Examples and features of embodiments of the MUDS are further described in PCT International Application No. PCT/US2016/012434, the disclosure of which is incorporated herein by reference in its entirety. The MUDS 130 may include one or more fluid reservoirs 132, such as one or more syringes. As used herein, the term “fluid reservoir” means any container capable of taking in and delivering a fluid, for example during a fluid injection procedure including, for example a syringe, a rolling diaphragm, a pump, a compressible bag, and the like. Fluid reservoirs may include the interior volume of at least a portion of a fluid pathway, such as one or more tubing lengths, that are in fluid communication with the interior of the fluid reservoir, including fluid pathway portions that remain in fluid communication with the fluid reservoir after the system is closed or fluidly isolated from the remainder of the fluid pathway. In some examples, the number of fluid reservoirs 132 may correspond to the number of bulk fluid sources 120. For example, with reference to FIG. 2, the MUDS 130 has three syringes 132 in a side-by-side arrangement such that each syringe 132 is fluidly connectable to one or more of the corresponding three bulk fluid sources 120. In some examples, one or two bulk fluid sources 120 may be connected to one or more syringes 132 of MUDS 130. Each syringe 132 may be fluidly connectable to one of the bulk fluid sources 120 by a corresponding bulk fluid connector 118 and an associated MUDS fluid path 134. MUDS fluid path 134 may have a spike element that connects to bulk fluid connector 118.

With reference to FIG. 2, the MUDS 130 is removably connectable to the housing 102 of the fluid injector system 100. As will be appreciated by one having ordinary skill in the art, it may be desirable to construct at least a portion of the MUDS 130 from a clear medical grade plastic in order to facilitate visual verification that a fluid connection has been established with the fluid injector system 100 or that air has been removed from the fluid reservoir. Visual verification is also desirable for confirming that no air bubbles are present within various fluid connections, for example after performing an air removal protocol, such as described herein. Various optical sensors (not shown) may also be provided to detect air either in the fluid lines or the fluid reservoir during a priming operation. Additionally, various lighting elements (not shown), such as light emitting diodes (LEDs), may be provided to actuate one or more optical sensors and indicate that sufficient air has been removed from the fluid reservoir.

With reference to FIG. 2, the MUDS 130 may include one or more valves 136, such as stopcock valves, for controlling which medical fluid or combinations of medical fluids are withdrawn from the multi-dose bulk fluid source 120 (see FIG. 1) into the fluid reservoirs 132 and/or are delivered to a patient from each fluid reservoir 132. In some examples, the one or more valves 136 may be provided on a distal end of the plurality of syringes 132 or on a manifold 148. The manifold 148 may be in fluid communication via valves 136 and/or syringes 132 with a first end of the MUDS fluid path 134 that connects each syringe 132 to the corresponding bulk fluid source 120. The opposing second end of the MUDS fluid path 134 may be connected to the respective bulk fluid connector 118 that is configured for fluidly connecting with the bulk fluid source 120. Depending on the position of the one or more valves 136, fluid may be drawn into the one or more syringes 132 or it may be delivered from the one or more syringes 132. In a first position, such as during the filling of the syringes 132, the one or more valves 136 are oriented such that fluid flows from the bulk fluid source 120 into the desired syringe 132 through a fluid inlet line 150, such as a MUDS fluid path. During the filling procedure, the one or more valves 136 are positioned such that fluid flow through one or more fluid outlet lines 152 or manifold 148 is blocked. In a second position, such as during a fluid delivery procedure, fluid from one or more syringes 132 is delivered to the manifold 148 through the one or more fluid outlet lines 152 or syringe valve outlet ports. During the delivery procedure, the one or more valves 136 are positioned such that fluid flow through one or more fluid inlet lines 150 is blocked. In a third position, the one or more valves 136 are oriented such that fluid flow through the one or more fluid inlet lines 150 and the one or more fluid outlet lines 152 is blocked. Thus, in the third position, each of the one or more valves 136 isolates the corresponding syringe 132 and prevents fluid flow into and out of the corresponding syringe 132. As such, each of the one or more syringes 132 and any portion of the manifold 148 between that syringe 132 and corresponding valve 136 defines a closed system. According to various aspects, the methods described herein may be used when fluid reservoir 132 is in the third, closed position

The one or more valves 136, fluid inlet lines 150, and/or fluid outlet lines 152 may be integrated into the manifold 148. The one or more valves 136 may be selectively positioned to the first or second position by manual or automatic handling. For example, the operator may position the one or more valves 136 into the desired position for filling, fluid delivery, or the closed position. In other examples, at least a portion of the fluid injector system 100 is operable for automatically positioning the one or more valves 136 into a desired position for filling, fluid delivery, or the closed position based on input by the operator or by a protocol in the system controller, as described herein.

In some examples, the fluid outlet line 152 may also be connected to a waste reservoir on the fluid injector system 100. In some examples, the waste reservoir is configured to receive waste fluid and air containing fluid expelled from the syringes 132 during, for example, a flushing, priming, air removal, or preloading operation.

Having generally described the components of the fluid injector system 100 and the MUDS 130, the structure and method of use of a single-use disposable set 190 (SUDS) and its interaction with MUDS 130 will now be described.

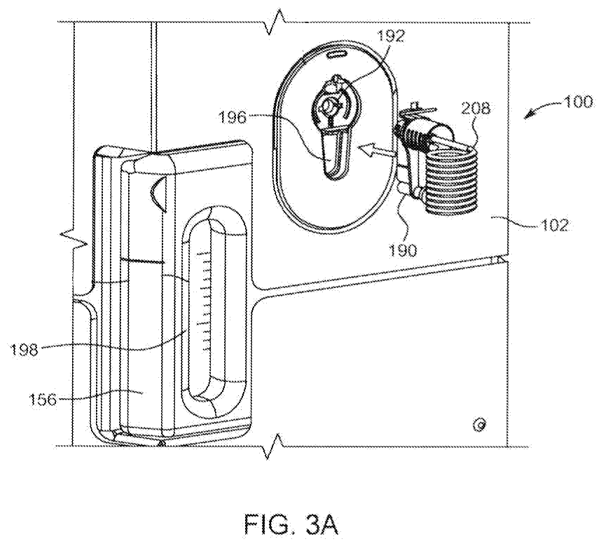

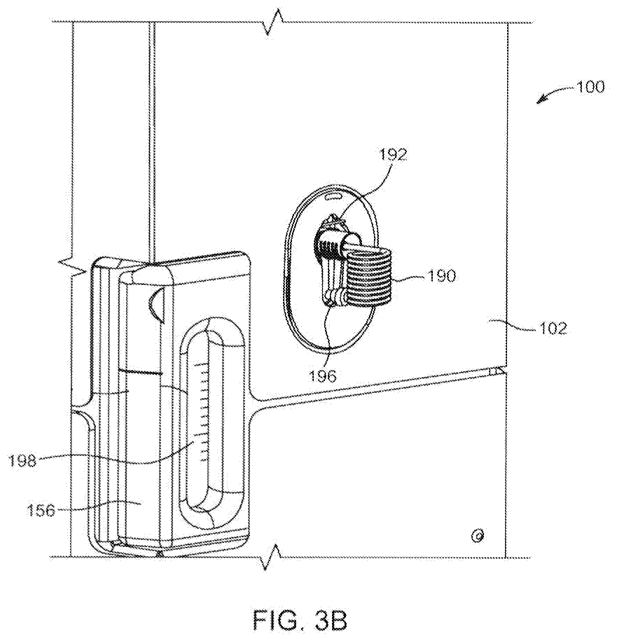

With reference to FIGS. 3A and 3B, the fluid injector system 100 has a connection port 192 that is configured to form a releasable fluid connection with at least a portion of the SUDS 190. In some examples, the connection port 192 may be formed on the MUDS 130. As described herein, the SUDS 190 may be connected to the connection port 192, formed on at least a portion of the MUDS 130 and/or the housing 102. Desirably, the connection between the SUDS 190 and the connection port 192 is a releasable connection to allow the SUDS 190 to be selectively disconnected from the connection port 192 (FIG. 3A) and connected to the connection port 192 (FIG. 3B). In some examples, the SUDS 190 may be disconnected from the connection port 192 and disposed after each fluid delivery procedure, and a new SUDS 190 may be connected to the connection port 192 for a subsequent fluid delivery procedure.

Other examples and features of the SUDS 190 are described in U.S. Patent Publication No. 2016/0331951, filed Jul. 7, 2016 and entitled “Single-Use Disposable Set Connector”, the disclosure of which is incorporated herein by reference in its entirety.

Having generally described the components of the fluid injector system 100, the MUDS 130, and the SUDS 190, a method of operation of using the SUDS 190 will now be described in detail. In use, a medical technician or user removes the disposable SUDS 190 from its packaging (not shown) and inserts the fluid inlet port 202 into the connection port 192 on the MUDS 130. The SUDS 190 may be secured to the MUDS 130 by inserting the locking tab 216 into the receiving slot 217 on the MUDS 130 and the controller determines that the SUDS 190 is securely connected to the MUDS 130, for example as sensed by the sensor 242. The fluid injector system 100 may perform an automatic priming or flushing operation for removing air from the MUDS 130 and the SUDS 190. Prior to or as part of the automatic priming and/or flushing operation, removal of additional air or gas bubbled adhered to an interior surface of the fluid reservoir 132 that are not may be removed during a bulk air priming or flushing operation may be removed according to various embodiments described herein. During such priming or flushing operations, fluid from the MUDS 130 including any air that was entrapped or present in the fluid reservoir 132 is injected through the connection port 192 and into the tubing 208 of the SUDS 190. The fluid flows through the tubing 208, the connector 214 and through the waste outlet port 204 and into the waste reservoir 156. Once the automatic priming or flushing operation is completed, the tubing 208 may optionally be preloaded by injecting fluid from the MUDS 130 through the connection port 192. After the automatic priming or flushing operation, including the air or gas removal processes described herein, and, optionally, the preloading operation are completed, the medical technician disconnects the connector 214 from the waste outlet port 204. The connector 214 may then be connected to the patient via a catheter, vascular access device, needle, or additional fluid path set to facilitate fluid delivery to the patient. Once the fluid delivery is completed, the SUDS 190 is disconnected from the patient and the MUDS 130 by disengaging the locking tab 216 of the SUDS 190 from the receiving slot 217 on the MUDS 130.

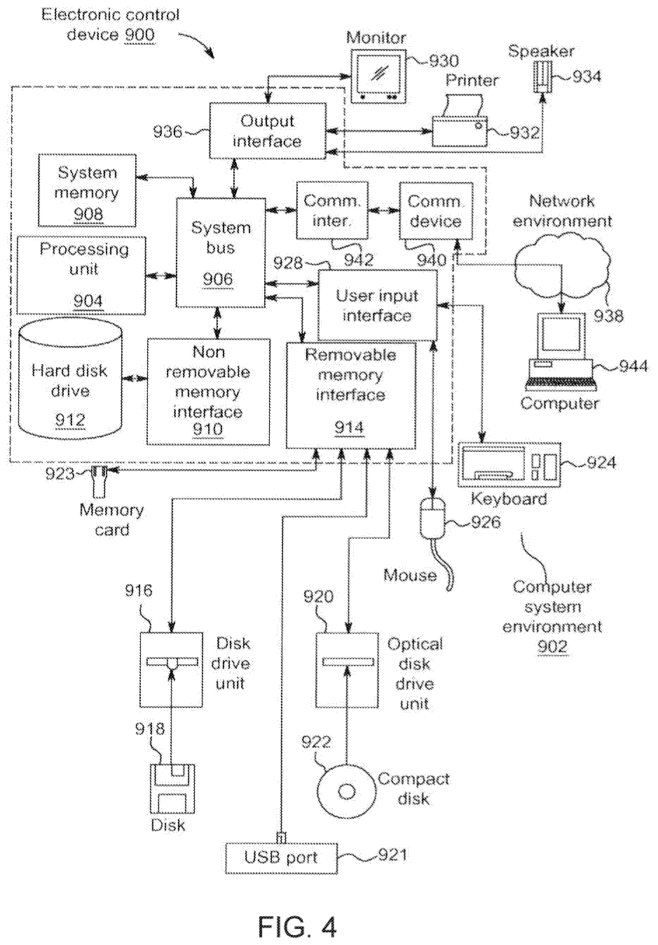

With reference to FIG. 4, an electronic control device 900 may be associated with fluid injector system 100 to control the filling and delivery operations. In some examples, the electronic control device 900 may control the operation of various valves, stopcocks, piston members, and other elements to affect a desired gas/air removal, filling, and/or delivery procedure. For example, the electronic control device 900 may include a variety of discrete computer-readable media components. For example, this computer-readable media may include any media that can be accessed by the electronic control device 900, such as volatile media, non-volatile media, removable media, non-removable media, transitory media, non-transitory media, etc. As a further example, this computer-readable media may include computer storage media, such as media implemented in any method or technology for storage of information, such as computer-readable instructions, data structures, program modules, or other data; random access memory (RAM), read-only memory (ROM), electrically erasable programmable read-only memory (EEPROM), flash memory, cloud storage media, or other memory technology; solid state memory, cloud memory, CD-ROM, digital versatile disks (DVDs), or other optical disk storage; magnetic cassettes, magnetic tape, magnetic disk storage, or other magnetic storage devices; or any other medium which can be used to store the desired information and which can be accessed by the electronic control device 900. Further, this computer-readable media may include communications media, such as computer-readable instructions, data structures, program modules, or other data in a modulated data signal, such as a carrier wave or other transport mechanism and include any information delivery media, wired media (such as a wired network and a direct-wired connection), and wireless media (such as acoustic signals, radio frequency signals, optical signals, infrared signals, biometric signals, bar code signals, etc.). Of course, combinations of any of the above should also be included within the scope of computer-readable media.

Electronic control device 900 may further include a system memory 908 with computer storage media in the form of volatile and non-volatile memory, such as ROM and RAM. A basic input/output system (BIOS) with appropriate computer-based routines assists in transferring information between components within electronic control device 900 and is normally stored in ROM. The RAM portion of system memory 908 may contain data and program modules that are immediately accessible to or presently being operated by a processor 904, e.g., an operating system, application programming interfaces, application programs, program modules, program data, or other instruction-based device-readable codes.

With continued reference to FIG. 4, the electronic control device 900 may also include other removable or non-removable, volatile or non-volatile, transitory or non-transitory computer storage media products. For example, the electronic control device 900 may include a non-removable memory interface 910 that communicates with and controls a hard disk drive 912, e.g., a non-removable, non-volatile magnetic medium; and a removable, non-volatile memory interface 914 that communicates with and controls a magnetic disk drive unit 916 (which reads from and writes to a removable, non-volatile magnetic disk 918), an optical disk drive unit 920 (which reads from and writes to a removable, non-volatile optical disk 922, such as a CD ROM), a Universal Serial Bus (USB) port 921 for use in connection with a removable memory card, etc. However, it is envisioned that other removable or non-removable, volatile or non-volatile computer storage media can be used in an exemplary computing system environment 902, including, but not limited to, magnetic tape cassettes, DVDs, digital video tape, solid state RAM, solid state ROM, etc. These various removable or non-removable, volatile or non-volatile magnetic media are in communication with the processor 904 and other components of the electronic control device 900 via a system bus 906. The drives and their associated computer storage media, discussed herein and illustrated in FIG. 4, provide storage of operating systems, computer-readable instructions, application programs, data structures, program modules, program data, and other instruction-based, computer-readable code for the electronic control device 900 (whether duplicative or not of this information and data in the system memory 908).

A user may enter commands, information, and data into the electronic control device 900 through certain attachable or operable input devices, such as the user interface 124 shown in FIG. 1, via a user input interface 928. A variety of such input devices may be utilized, e.g., a microphone, a trackball, a joystick, a touchpad, a touch-screen, a scanner, etc., including any arrangement that facilitates the input of data and information to the electronic control device 900 from an outside source. As discussed, these and other input devices are often connected to the processor 904 through the user input interface 928 coupled to the system bus 906, but may be connected by other interface and bus structures, such as a parallel port, game port, or a USB. Still further, data and information can be presented or provided to a user in an intelligible form or format through certain output devices, such as a monitor 930 (to visually display this information and data in electronic form), a printer 932 (to physically display this information and data in print form), a speaker 934 (to audibly present this information and data in audible form), etc. All of these devices are in communication with electronic control device 900 through an output interface 936 coupled to system bus 906. Any such peripheral output devices may be used to provide information and data to the user.

Electronic control device 900 may operate in a network environment 938 through the use of a communications device 940, which is integral to electronic control device 900 or remote therefrom. Communications device 940 is operable by and in communication with the other components of the electronic control device 900 through a communications interface 942. Using such an arrangement, the electronic control device 900 may connect with or otherwise communicate with one or more remote computers, such as a remote computer 944, which may be a personal computer, a server, a router, a network personal computer, a peer device, or other common network nodes, and typically includes at least some of the components described in connection with the electronic control device 900. Using appropriate communication devices 940, e.g., a modem, a network interface or adapter, etc., computer 944 may operate within and communicate through a local area network (LAN) and a wide area network (WAN), but may also include other networks such as a virtual private network (VPN), an office network, an enterprise network, an intranet, the Internet, etc.

As used herein, the electronic control device 900 includes or is operable to execute appropriate custom-designed or conventional software to perform and implement the processing steps of the method and system of the present disclosure, thereby forming a specialized and particular computing system. Accordingly, the method and system may include one or more electronic control devices 900 or similar computing devices having a computer-readable storage medium capable of storing computer-readable program code or instructions that cause the processor 904 to execute, configure, or otherwise implement the methods, processes, and transformational data manipulations discussed hereinafter in connection with the present disclosure. Still further, the electronic control device 900 may be in the form of a personal computer, a personal digital assistant, a portable computer, a laptop, tablet, a palmtop, a mobile device, a mobile telephone, a server, or any other type of computing device having the necessary processing hardware to appropriately process data to effectively implement the computer-implemented method and system.

It will be apparent to one skilled in the relevant arts that the system may utilize databases physically located on one or more computers which may or may not be the same as their respective servers. For example, programming software on electronic control device 900 can control a database physically stored on a separate processor of the network or otherwise.

In some examples, the electronic control device 900 may be programmed so that automatic refill occurs based upon a preprogrammed trigger minimum volume in the respective syringes 132. For example, when the volume of fluid remaining in at least one of the syringes 132 is less than a programmed volume, a syringe refill procedure is automatically initiated by the electronic control device 900. The electronic control device 900 associated with the fluid injector system 100 may determine that the preprogrammed trigger minimum volume has been reached by tracking the fluid volume dispensed from the respective syringes 132 during operation of the fluid injector system 100. Alternatively, fluid level sensors may be incorporated into the fluid injector system 100 and inputs from these fluid level sensors may be provided to the electronic control device 900 so that the electronic control device 900 may determine when the preprogrammed trigger minimum volume has been reached in at least one of the syringes 132. The fill volume and rate of refill can be preprogrammed in the electronic control device 900. The automatic refill procedure can be stopped either automatically by the electronic control device 900 or may be manually interrupted. In addition, an automatic refill procedure may be initiated when, at the completion of a fluid injection procedure, there is not enough fluid in at least one of the syringes 132 to perform the next programmed fluid injection procedure. Once the automatic refill has been triggered, the fluid injector may run the air removal protocol according to embodiments described herein.

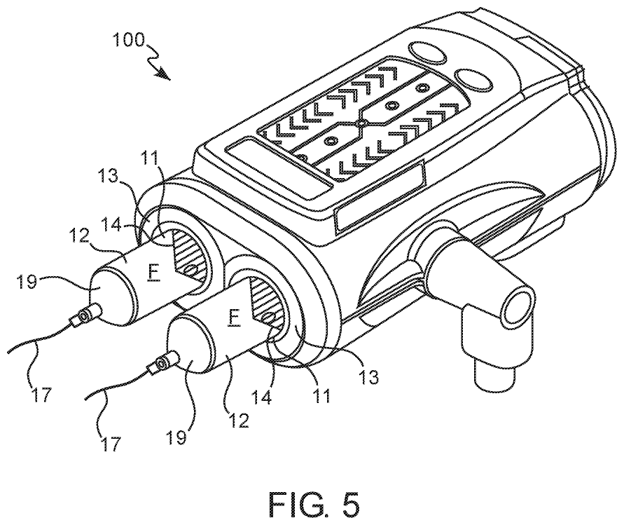

While FIGS. 1-3B illustrate one example of a fluid injector system 100 and associated components and structure, it is to be understood that the present disclosure is not limited to any particular type or variety of the fluid injector system 100. Referring now to FIG. 5, another example of a fluid injector system 100 According to the present disclosure includes at least one fluid reservoir, such as syringe 12, at least one piston 103 (not shown) connectable to at least one plunger 14, and a fluid control module (not pictured). The at least one syringe 12 is generally adapted to interface with at least one component of the system, such as a syringe port 13. The fluid injector system 100 is generally configured to deliver at least one fluid F to a patient during an injection procedure. The fluid injector system 100 is configured to releasably receive the at least one syringe 12, which is to be filled with at least one fluid F, such as contrast media, saline solution, or any desired medical fluid. The system may be a multi-syringe injector, wherein several syringes may be oriented side-by-side or in another spatial relationship and are separately actuated by respective pistons associated with the injector. The at least one syringe 12 may be oriented in any manner such as upright, downright, or positioned at any degree angle.

With continued reference to FIG. 5, the injector system 100 may be used during a medical procedure to inject the medical fluid F into the body of a patient by driving a plunger 14 of at least one syringe 12 with a drive member, such as the at least one piston 103 (not shown). The at least one piston may be reciprocally operable upon at least a portion of the at least one syringe, such as the plunger 14. Upon engagement, the at least one piston may move the plunger 14 toward the distal end 19 of the at least one syringe, as well as toward the proximal end 11 of the at least one syringe 12.

A tubing set 17 may be in fluid communication with each syringe 12 to place each syringe in fluid communication with a catheter for delivering the fluid F from each syringes 12 to the catheter (not shown) inserted into a patient at a vascular access site. In certain embodiments, fluid flow from the one or more syringes 12 may be regulated by a fluid control module, which may be the same or similar to the electronic control device 900, that operates various valves, stopcocks, and flow regulating structures to regulate the delivery of the at least one fluid to the patient based on user selected injection parameters, such as injection flow rate, duration, and total injection volume. The fluid control module is generally configured to perform various functions, those of which have the ability to aid in the removal of gas from the system, as will be described herein, along with other various embodiments.

In some examples, the fluid control module may instruct the fluid injector system 100 to fill the at least one syringe 12 with the at least one fluid F. The fluid injector system 100 may further include at least one bulk fluid source (not shown) for filling the at least one syringe 12 with fluid and in certain examples, the fluid injector system 100 may have a plurality of bulk fluid sources, one for each of the syringes 12, for filling each of the syringes with the desired fluid. Filling the at least one syringe 12 with the at least one fluid F may be done by placing the at least one syringe 12 in fluid communication with at least one bulk fluid source and instructing the fluid injector system 100 to withdraw the piston, being removably engaged with the plunger 14 of the at least one syringe 12, from the distal end 19 of the at least one syringe toward the proximal end 11 of the at least one syringe. Filling in such a manner provides that the at least one syringe may be oriented in any manner during the filling procedure, such as upwards, downwards, or at any degree angle. In certain embodiments, the fluid injector system 100 and fluid control module may be programmed to perform an air removal protocol, as described herein. In certain embodiments, the air removal protocol may be performed on an open system, i.e., the at least one syringe 12 may be in fluid connection with the tubing set 17 and the protocol may utilize one or more of the viscosity of the fluid, the flow rate, the difference between the diameter of the syringe and the flow path tubing to generate at least a partial vacuum, for example by quickly retracting the piston and plunger at a designated rate, to affect the air removal protocol as described herein. According to other embodiments, the distal end 19 of the syringe 12 and/or the tubing set 17 may include one or more valves or stop cocks to fluidly isolate the interior of the syringe from the outside to allow the injector system 100 to generate the at least partial vacuum sufficient to affect the air removal protocol, as described herein.

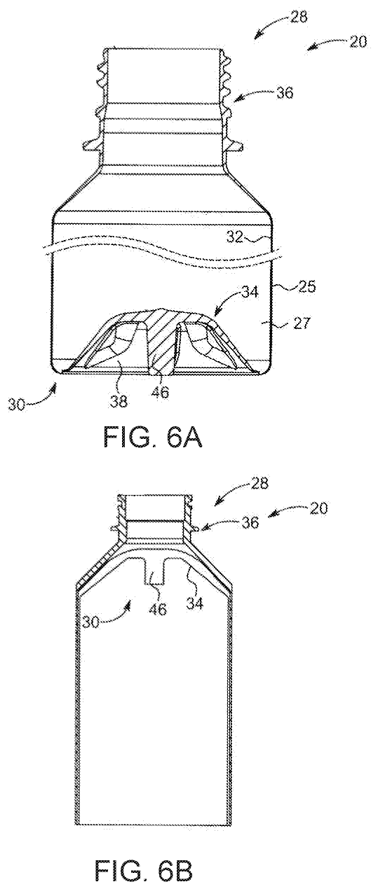

The examples of fluid injector systems 100 described in connection with FIGS. 1-5 are generally illustrated as having rigid-bodied syringes 132, 12. However, it is to be understood that the present disclosure contemplates that other fluid reservoirs and varieties of syringes may be utilized in the fluid injector systems 100 described herein. Referring now FIGS. 6A and 6B, in some examples a rolling diaphragm the syringe 20 generally includes a hollow body 25 defining an interior volume 27. The body 25 has a forward or distal end 28, a rearward or proximal end 30, and a flexible sidewall 32 extending therebetween. The sidewall 32 of the syringe 20 defines a soft, pliable or flexible, yet self-supporting body that is configured to roll upon itself, as a rolling diaphragm, under the action of the piston 19. In particular, the sidewall 32 is configured to roll such that its outer surface is folded and inverted in a radially inward direction as the piston 103 is moved in a distal direction (FIG. 6B) and unrolled and unfolded in the opposite manner in a radially outward direction as the piston 103 is retracted in a proximal direction (FIG. 6A).

The sidewall 32 may have a smooth, substantially uniform structure, or it may have one or more ribs provided thereon to facilitate the rollover during an injection procedure. According to these embodiments, the end wall 34 may have a piston engagement portion 46 located on a proximal end of the plunger to interact with the plurality of engagement elements of the various embodiments of the engagement mechanisms on a piston of the fluid injector. Examples of rolling diaphragm syringes and injector piston configurations suitable for use in the air removal protocols of the present disclosure are described in U.S. Application Publication Nos. 2017/0035974; and 2018/0261496; and in PCT International Application Publication Nos. WO 2018/075379; and WO 2018/075386, the disclosures of which are hereby incorporated by reference in their entireties.

With continued reference to FIG. 6A-6B, the rearward or proximal portion 30 of the sidewall 32 connects to a closed end wall 34, and a forward or distal portion 28 of the sidewall 32 defines a discharge neck 36 opposite the closed end wall 34. The closed end wall 34 may have a concave shape to facilitate the initiation of the inversion or rolling of the sidewall 32 and/or to provide a receiving pocket to receive a distal end of the piston 19. For example, the closed end wall 34 may define a receiving end pocket 38 for interfacing directly with a similarly-shaped piston 19.

Having described various aspects of the fluid injector system 100, embodiments for processor programing and methods of gas removal from at least one fluid reservoir of the fluid injector system 100 will now be described. The at least one fluid reservoir may be as described herein and may, for example, include at least one of the syringes 132, 12 and/or the rolling diaphragm syringe 20. Alternatively or in additionally, the at least one reservoir may include at least one bottle, or at least one collapsible bag. One of ordinary skill in the art would appreciate that this list is merely exemplary and the method of gas removal may extend to other reservoirs. Further, it should be appreciated that the method of gas removal can be performed on a fluid injector system of any fluid volume, such as for example on a fluid reservoir of 0.1 milliliters (mL) through 1000 mL and in other embodiments from 10 mL to 300 mL. As will be described in greater detail herein, the methods of gas removal According to the present disclosure relate to both closed and open fluid injector systems 100, i.e., a fluid injector system where the one or more fluid reservoirs (optionally including at least a portion of the tubing set) may be in fluid communication with or fluidly isolated from the remainder of the fluid flow path of the system, including embodiments where at least one of the fluid reservoirs is in fluid isolation and at least one of the other reservoirs may be in fluid communication with the system.

In one example of the present disclosure, a method of gas removal may be utilized in a closed or closeable fluid injector system 100, meaning that the fluid injector system 100 has at least one state in which the at least one fluid reservoir (optionally including at least a portion of the tubing set) is not exposed to atmospheric pressure and/or is fluidly isolated from the remainder of the fluid injection flow path. For example, with reference to the fluid injector system 100 of FIGS. 1-3B, each of the syringes 132 may be isolated from atmospheric pressure by rotating the associated valve 136 to a closed position to prevent fluid flow into and from the syringe 132. The fluid injector system 100 of FIG. 5 may also be converted into a closed or closeable system by placement of an isolation valve, such as a pinch valve, a shuttle valve, a check valve, high pressure crack valve, or a stopcock, in any of the fluid path sets 17 or between any of the fluid path sets 17 and the associated syringe 12. One of ordinary skill in the art would appreciate that this list is merely exemplary and other types of isolation valves may be used.

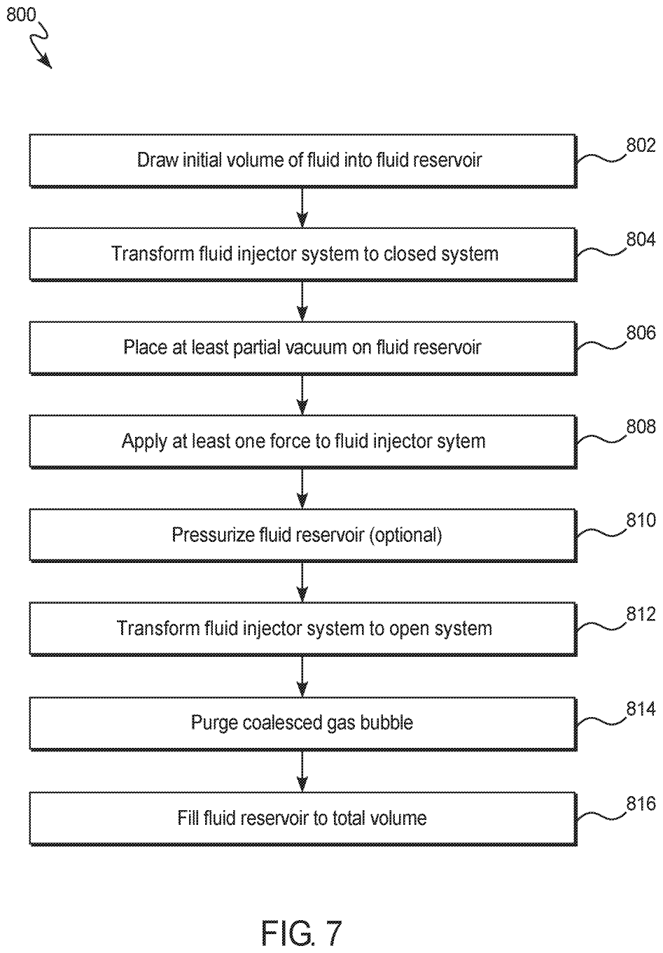

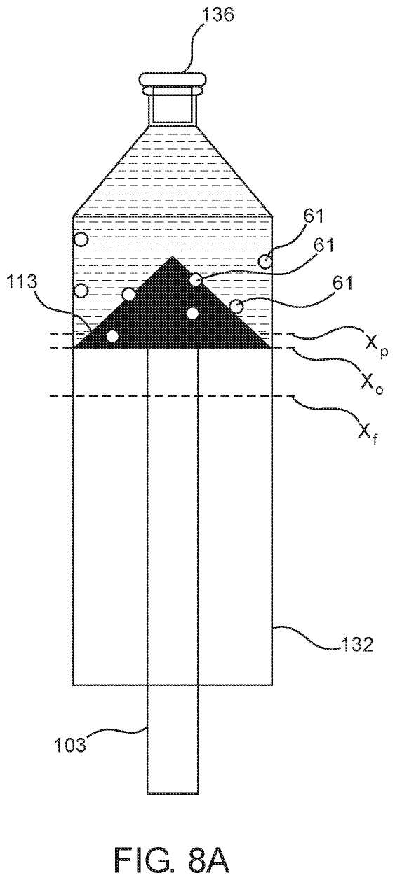

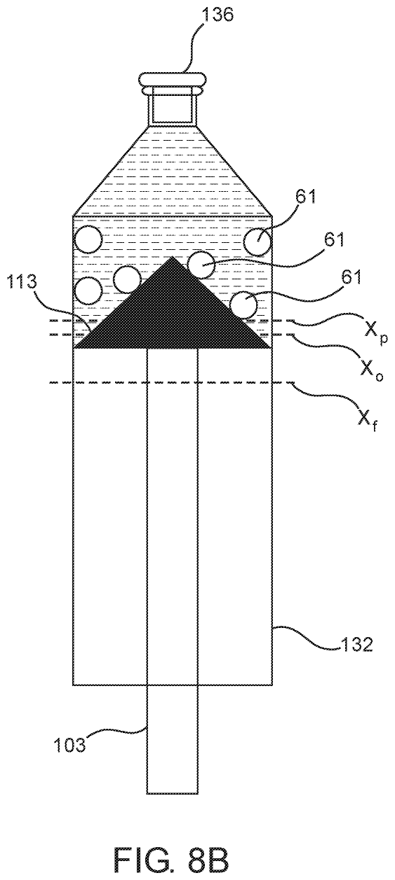

Referring now to FIGS. 7 and 8A-8C, a method 800 of gas removal from a closed or closeable system will be described. For simplicity, the method 800 will be described primarily with respect to the example of the fluid injector system 100 described in connection with FIGS. 1-3B. However, the method 800 may also be used with other fluid reservoir based fluid injector systems having a closable configuration that fluidly isolates the reservoir from the atmosphere, for example the fluid injector system 100 and associated syringes 12 illustrated in FIG. 5, having first converted the fluid injector system 100 of FIG. 5 to a closeable system, or with a rolling diaphragm-based fluid injection system, as described herein. At step 802, an initial volume of fluid is drawn into at least one fluid reservoir, e.g. at least one syringe 132, of the fluid injector system 100. The valve 136 associated with the at least one syringe 132 is placed in a position allowing fluid flow into the syringe 132 from a fluid source in fluid communication with the syringe 132, such as one or more of the bulk fluid sources 120. A plunger 113 positioned at a distal or near distal location with the syringe 132 may be moved proximally within the syringe 132 to draw an initial volume of fluid into the syringe 132. In some examples, the plunger 113 may be removably connected to a distal end of the piston 103, and the piston 103 may be actuated by the electronic control module 900 to move the plunger 113 within the syringe 132. The initial volume of fluid drawn into the syringe 132 may be, for example, from 10 mL to 300 mL. The initial volume of fluid drawn into the syringe 132 may include air or other gas in a significant volume, the majority of which may be removed by a purge or prime sequence, and/or may have a clinically insignificant amount of air or gas adhered to one or more interior surfaces of the fluid reservoir, for example as small but visible bubbles. According to various embodiments, the small but visible bubbles may remain adhered to the one or more interior surfaces after a prime/purge sequence due to their size and/or buoyancy not being sufficient to overcome surface tension or adhesion between the bubble and the one or more interior surfaces. While potentially clinically insignificant, the gas may nevertheless be manifested as bubbles 61, which may be visible to the patient and the technician through the transparent sidewall of the syringe 132. In other procedures such as an angiography procedure, even small amounts of air or gas may be clinically significant and must be removed from the fluid reservoir prior to initiation of the injection procedure. The small bubbles 61 may particularly adhere to at least one interior surface of the syringe 132, such as the sidewall of the syringe 132 and/or a surface of the plunger 113. The bubbles 61 may manifest in a variety of sizes, with particularly large air bubbles 61 being up to 5 to 20 mL in volume or consisting of up to 10% of the volume of the syringe 132. Smaller air bubbles 61 may be 0.001 to 5 mL in volume or consist of up to 2.5% of the volume of the syringe 132 may also be removed according to the present disclosure. Larger air bubbles may have sufficient buoyancy to overcome surface tension and adhesive forces with the interior surfaces and float to the distal end of the fluid reservoir to be removed during priming operations. However, as described herein, smaller air bubbles 61 may remain adhered to the interior surfaces during the priming/purging sequences, but may be removed utilizing the methods described herein.

With continued reference to FIG. 7, at step 804, the fluid injector system 100 is transformed to a closed system, such that the at least one fluid reservoir is isolated from atmospheric pressure for example by closing an isolation valve. In some examples, the electronic control device 900 may be configured to transform the fluid injector system 100 from an open system to a closed system by rotating or otherwise adjusting the valve 136 to isolate the at least one fluid reservoir, e.g. at least one syringe 132, from atmospheric pressure. In other examples, the valve 136 may be manually rotated to isolate the at least one syringe 132 from atmospheric pressure by a technician or a physician.

With continued reference to FIGS. 7 and 8A-8B, at step 806, an at least partial vacuum may be placed on the at least one fluid reservoir, e.g. the at least one syringe 132. The at least partial vacuum within the at least one fluid reservoir may be achieved by expanding the volume of the closed system, resulting in a pressure drop and thus creation of the at least partial vacuum. In some embodiments, the at least partial vacuum may be achieved by creating a displacement within the fluid injector system 100, for example by reciprocally retracting the plunger 113 of the at least one syringe 132 by actuating the piston 103 via the electronic control module 900. In some examples, the plunger 113 may be moved via the piston 103 at a constant speed, for example in a range of approximately 0 mL/s to 50 mL/s. In some examples, movement of the piston 103 may be programmed into the electronic control module 900, such that the plunger 113 may be retracted automatically or may be initiated by a technician or physician by entering a command via the user interface 124. In other examples, the technician or physician may manually control the piston 103 and the plunger 113 via the user interface 124. With reference to FIGS. 8A-8C, the plunger 113 may be moved from a starting position x₀ to a final ending position x_{f}. The starting position x₀ may represent a position within the syringe 132 located distally relative to the final ending position x_{f}. For example, the starting position x₀ may be a position within the syringe 132 corresponding to the 100 mL initial volume of fluid drawn into the syringe 132, and the final ending position x_{f} may be a position of the syringe 132 that represents 120 mL, thus creating a displacement of 20 mL of the plunger 113. Other final ending positions x_{f} may be used depending on the vacuum pressure required to remove the amount of small air bubbles desired, according to the methods herein.

In some examples, the at least partial vacuum placed on the at least one fluid reservoir may be automated. In some examples, the electronic control module 900 may be configured to automatically generate the at least partial vacuum by actuating the piston 103 and drawing in the proximal direction after the initial volume of fluid is drawn into the syringe 132 and the valve 136 is closed in steps 802-804. In other examples, the electronic control module 900 may be configured to automatically generate the at least partial vacuum by actuating the piston 103 upon detection of gas bubbles 61 remaining in the volume of fluid drawn into the syringe at step 802, for example after a bulk air purge process. In other examples, the electronic control module 900 may be configured to generate the at least partial vacuum by actuating the piston 103 upon manual entry of a command into the user interface 124 by the technician or physician. In certain embodiments, the control module 900 may be configured to determine the amount of adhered gas bubbles on the interior surfaces of the fluid reservoir, and utilize an algorithm or look-up table based on the amount of adhered gas bubbles to determine and implement the minimum at least partial vacuum necessary to effect removal of the adhered gas bubbles using the methods described herein.

With continued reference to step 806, the gas bubbles 61 may be dislodged from the interior surface of the fluid reservoir, for example syringe 132, due to the creation of the at least partial vacuum. With reference to FIG. 8B, as a consequence of the change in pressure (ΔP) generated within the syringe 132, the pressure within the fluid and gas contents of the closed system, including the pressure within each bubble 61, is reduced which causes each bubble 61 to expand in volume and increase in overall surface area and buoyancy. Moreover, the proportion of the surface area of each bubble 61 in contact with the at least one interior surface of the syringe 132 may be reduced due to the volumetric expansion of each bubble 61. Referring now to FIG. 8C, the increased buoyancy and reduced contact area with the at least one interior surface of the syringe 132 of each bubble may overcome the adhesion forces between the bubbles 61 and the at least one interior surface of the syringe 132, inducing the bubbles 61 to dislodge from the at least one interior surface of the syringe 132 and float to the distal end or highest point of the syringe 132. As the bubbles 61 rise to the distal end or highest point in the syringe 132, the bubbles 61 may develop an affinity to coalesce with one another and form a coalesced bubble 62 of larger volume. The coalesced bubble 62 may have a reduced effective surface area in comparison to the combined effective surface areas of the individual bubbles 61. The reduction in effective surface area may in turn minimize the total surface tension and surface energy associated with coalesced bubble 62.

It is noted that while FIG. 8B shows the expansion of the bubbles 61 and FIG. 8C shows the coalescence of the bubbles 61 into the coalesced bubble 62, the expansion and coalescence of the bubbles 61 may in reality not be discrete steps, but may rather occur gradually and at least partially simultaneously as the at least partial vacuum is generated within the syringe 132. According to certain embodiments, if sufficient vacuum, as determined by the ΔP, is generated within the syringe 132, the reduced pressure may also draw dissolved gases out of the fluid which may coalesce with the coalesced bubble 62.

With continued reference to FIGS. 7-8C, at optional step 808, dislodging the gas bubbles 61 from the at least one interior surface of the fluid reservoir may be assisted by applying at least one force to the fluid injector system 100. In one example, this force may encompass a single or multiple impacts to at least one portion of the fluid injector system at or near the final ending position x_{f} while the fluid in the at least one fluid reservoir of an injector system 100 is under an at least partial vacuum. The single or multiple impacts may be made to the syringe 132 via actuation of the piston 103 or by a separate mechanism that creates one or more impacts to the fluid injector system 100. The single impact may also be made to any portion of the fluid path set in fluid communication with the fluid filled reservoir, such as the manifold 148. In another example, the at least one force may include at least one vibration, performed at a specific or randomized frequency and amplitude, of the fluid injector system 100. The at least one vibration may be created by a vibrational actuator 68 that produces an oscillating force across a single axis, such as a linear resonant actuator or an eccentric rotating mass motor. The vibrational actuator 68 may be located along at least one portion of the fluid injector system 100, such as at a portion of the outer surface of the fluid filled reservoir or a portion of the outer surface of the any portion of the fluid path set, such as the manifold 148. In another example, the vibrational actuator 68 may be located on or within the piston 103. The vibrational actuator, wherever located, ultimately causes vibration of the surfaces of the fluid reservoir, the surfaces of at least a portion of the fluid path set in fluid communication with the fluid reservoir, and/or the fluid within the syringe 132. In other examples, the fluid reservoir may be vibrated by pulsatile movements of the piston 103. In this example, the electronic control module 900 may instruct the piston 103 to pulsate, e.g., rapidly move by small displacements in the distal and proximal directions, thereby moving the plunger 113, which in turn may produce vibrations of the fluid within the syringe 132. As noted above, step 808 is optional and may be omitted in some examples of the present disclosure. According to these embodiments, the application of the at least one force to the fluid injector system 100 may dislodge one or more of the gas bubbles 61, for example by decreasing the contact surface area between the gas bubble 61 and the interior surface and allowing the buoyancy of the gas bubble 61 to overcome the surface tension adhesive forces of the reduced surface area of contact.

With continued reference to FIG. 7, at optional step 810, the fluid reservoir may be pressurized to ensure that the coalesced bubble 62 remains coalesced and is forced to a distal-most region of the syringe 132. Further, under the increased pressure, the coalesced bubble 62 is immediately moved down the fluid path when the valve 136 is moved to the open position (at step 812), such that the coalesced bubble 62 is transferred to the bulk fluid reservoir or purged from the system as the valve 136 is opened. Pressurization of the coalesced bubble 62 may be achieved by moving the plunger 113 distally in the syringe 132 via the piston 103 while the fluid injector system 100 is in the closed position. For example according to a non-limiting embodiment, if during step 806 the plunger 113 is proximally displaced 20 mL (e.g. from the starting position x₀ corresponding to 100 mL to the final ending position x_{f} corresponding to 120 mL), the plunger 113 may then be moved distally 21 mL, to a position xₚ corresponding to 99 mL within the syringe 132, to pressurize the coalesced bubble 62. Pressurization of coalesced bubble 62 may be performed automatically by the electronic control module 900. For example, the electronic control module 900 may be programmed or configured to wait a predetermined amount of time after generating the at least partial vacuum at step 806 to ensure that the individual bubbles 61 have had sufficient time to coalesce into the coalesced bubble 62. After the predetermined amount of time has elapsed, the electronic control module 900 may automatically instruct the piston 103 to move distally to position xₚ to pressurize the coalesced bubble 62. In other examples, pressurization of the coalesced bubble 62 may be performed manually via the technician or physician entering a command into the user interface 124.

With continued reference to FIG. 7, at step 812, the fluid injector system 100 is transformed into an open system, such that the at least one fluid reservoir is in fluid communication with atmospheric pressure. In some examples, the electronic control device 900 may be configured to transform the fluid injector system 100 from a closed system to an open system opening, for example by rotating or otherwise adjusting, the valve 136 to place the at least one fluid reservoir, e.g. the at least one syringe 132, in fluid communication with atmospheric pressure, for example either in fluid communication with the fluid path to the bulk fluid containers or in fluid communication with the tubing set that will be connected to the patient after gas removal. In other examples, valve 136 may be manually rotated by a technician or a physician to place the at least one syringe 132 in fluid communication with atmospheric pressure.

With continued reference to FIG. 7, at step 814, the coalesced bubble 62 is purged from the at least one fluid reservoir once the fluid injector system 100 is transformed into an open system. In some examples, the electronic control module 900 may actuate the piston 103 to move the plunger 113 distally in the syringe 123 such that the coalesced bubble 61 is expelled from the fluid path set downstream of the syringe 132, such as the manifold 148 and/or the one or more fluid outlet lines 152 or into the bulk fluid reservoir. In some examples, purging the coalesced bubble 62 from the syringe 132 may be achieved by distally advancing the plunger 113, via the piston 103, a predetermined distance according to an estimated volume of the coalesced bubble 62 within the syringe 132 and the volume of the fluid path that the bubble travels through while being purged, such as, for example, 20 mL. Alternatively, if optional step 810 is performed to pressurize the coalesced bubble 62, the total piston movement distance may be reduced. In some examples, coalescing any adhered bubbles 61 and purging the coalesced bubble 62 may be performed automatically as part of a filling, priming, or injection procedure. In some examples, the electronic control module 900 may instruct the piston 103 to purge the coalesced bubble 62 prior to initiating a diagnostic injection protocol. In other examples, purging of the coalesced bubble 62 may be performed manually via the technician or physician entering a command into the user interface 124.

With continued reference to FIG. 7, at step 816, the fluid reservoir is filled to a total volume (i.e. 100% volume) of fluid prescribed by the diagnostic injection protocol to account for the volume lost during purging of the coalesced bubble 62. In some examples, the filling may be achieved by proximally retracting plunger 113, via piston 103, a predetermined distance, corresponding to a predetermined volume, within syringe 132. At this stage, the fluid reservoir may be filled with the volume of fluid necessary to complete the diagnostic injection protocol and substantially all of the visible gas has been purged. As such, the fluid reservoir is prepared for fluid injector system 100 for the diagnostic injection protocol.

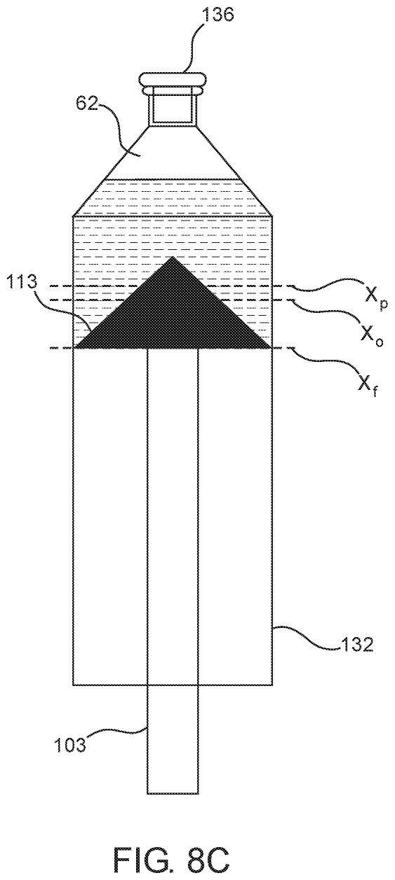

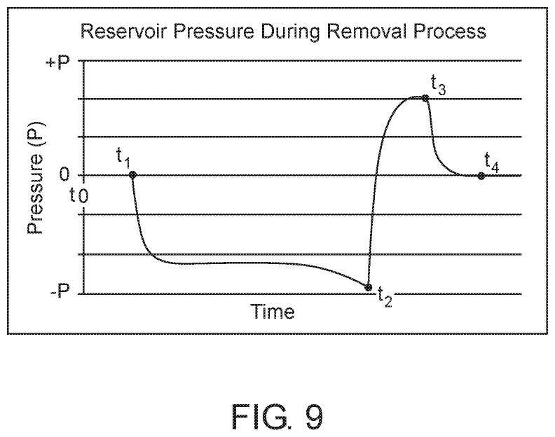

A graphical representation of the pressure within the fluid reservoir as a function of time during performance of one embodiment of the method 800 is shown in FIG. 9. Pressure (P) is shown on the y-axis of FIG. 9, with (+P) indicating a positive pressure relative to an atmospheric pressure, e.g. atmospheric pressure within the fluid source 120, and (−P) indicating a reduction in pressure, or vacuum pressure, relative to an atmospheric pressure, e.g. atmospheric pressure within the fluid source 120. Time (t) is shown on the x-axis of FIG. 9 and increases from an initial time t₀ to a final time t_{f}. At time t₀, the pressure (P) within the fluid reservoir is approximately equal to the atmospheric pressure of a component, e.g. the fluid source 120, to which the fluid reservoir is in fluid communication. At some time between time t₀ and t₁, the initial volume of fluid is drawn into the fluid reservoir and the injector system is transformed into a closed system, as described herein with reference to steps 802-804 of the method 800. At time t₁, an at least partial vacuum is placed on the fluid reservoir, as described herein with reference to step 806. As such, the pressure (P) within the fluid reservoir decreases from time t₁ to time t₂ until a maximum negative pressure (i.e. a maximum vacuum) is reached at time t₂. At time t₂, the vacuum is relieved and the fluid reservoir is pressurized as describe herein with reference to step 810. At time t₃, once the desired pressurization of the fluid reservoir is attained, the fluid reservoir is at a maximum positive pressure. At time t₃, the fluid injector system is transformed into an open system, as described herein with reference to step 812, causing the pressure within the fluid reservoir to be relieved. The pressure within the fluid reservoir decreases from the maximum pressure at time t₃ to atmospheric pressure at time t₄, as the coalesced bubble is purged from the fluid reservoir as described herein with reference to step 814. After time t₄, filling of the fluid reservoir according to step 816 may be performed, followed by performance of the injection protocol. It is noted that the graphical representation illustrated in FIG. 9 is merely exemplary of certain embodiments of the method 800 and may not be shown to scale.

In some examples of the present disclosure, the method 800, as performed by the fluid injector system 100, may be implemented by a computer program product. The computer program product may include at least one non-transitory computer-readable medium having one or more instructions executable by at least one processor to cause the at least one processor to execute all or part of the method 800. In some examples or aspects, the at least one non-transitory computer-readable medium and the at least one processor may include or correspond to the memory 908 and processor 904, respectively, as described above with reference to FIG. 4.

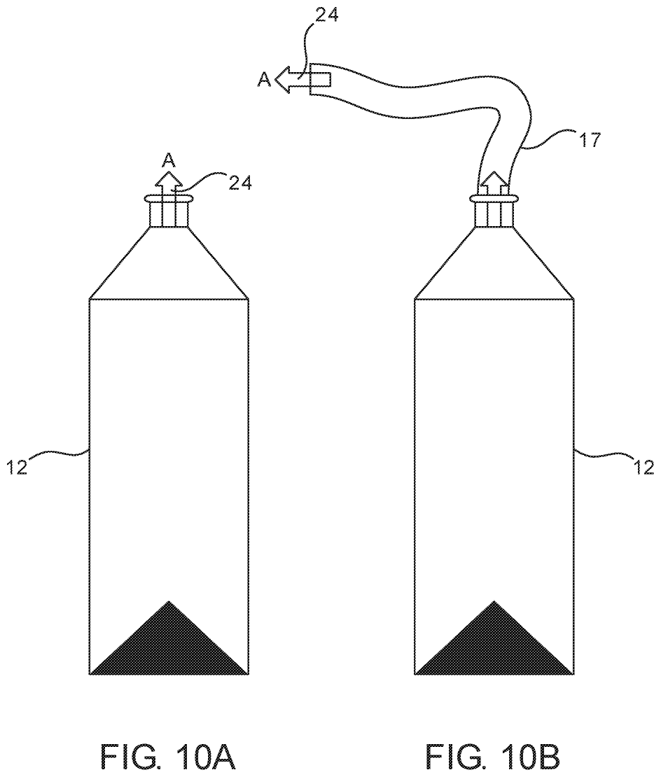

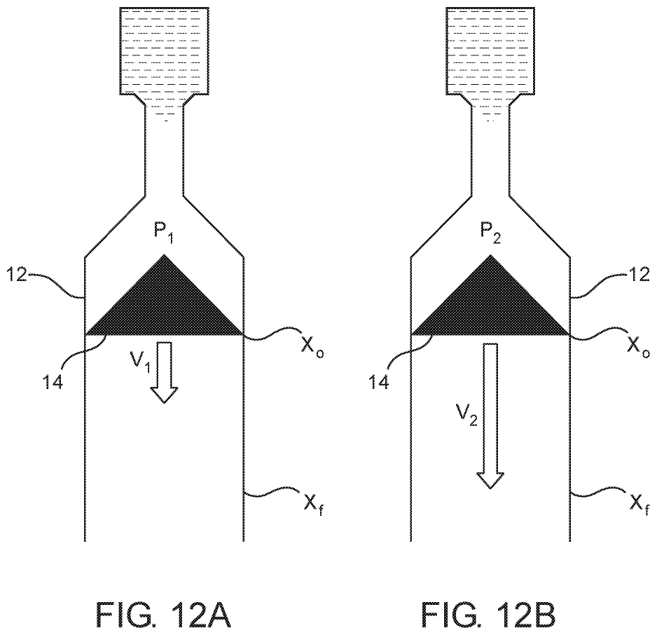

In other examples of the present disclosure, a method 850 of gas removal may be utilized in an open fluid injector system 100, meaning that the at least one fluid reservoir of the fluid injector system 100 remains in fluid communication with atmospheric pressure throughout the steps of the method 850. For simplicity, embodiments of the method 850 may be described primarily with respect to the example of the fluid injector system 100 described in connection with FIGS. 5, 10A,B, and 12A,B although the process may be utilized by other open fluid injection systems. FIGS. 10A,B and 12A,B show manners in which the fluid reservoirs, e.g. the syringes 12 of the fluid injector system 100 of FIG. 5, may be in fluid communication with atmospheric pressure A, such as atmospheric pressure A of the fluid source. In FIG. 10A, an outlet 24 of the syringe 12 is in direct fluid communication with atmospheric pressure A. In FIG. 12B, the outlet 24 of the syringe 12 is in fluid communication with atmospheric pressure A via the fluid path set 17. The method 850 may also be used with examples of the fluid injector system 100 of FIGS. 1-3B, provided that the valves 136 thereof are positioned to maintain fluid communication with atmospheric pressure throughout the steps 858-876 (described herein) of the method 850.

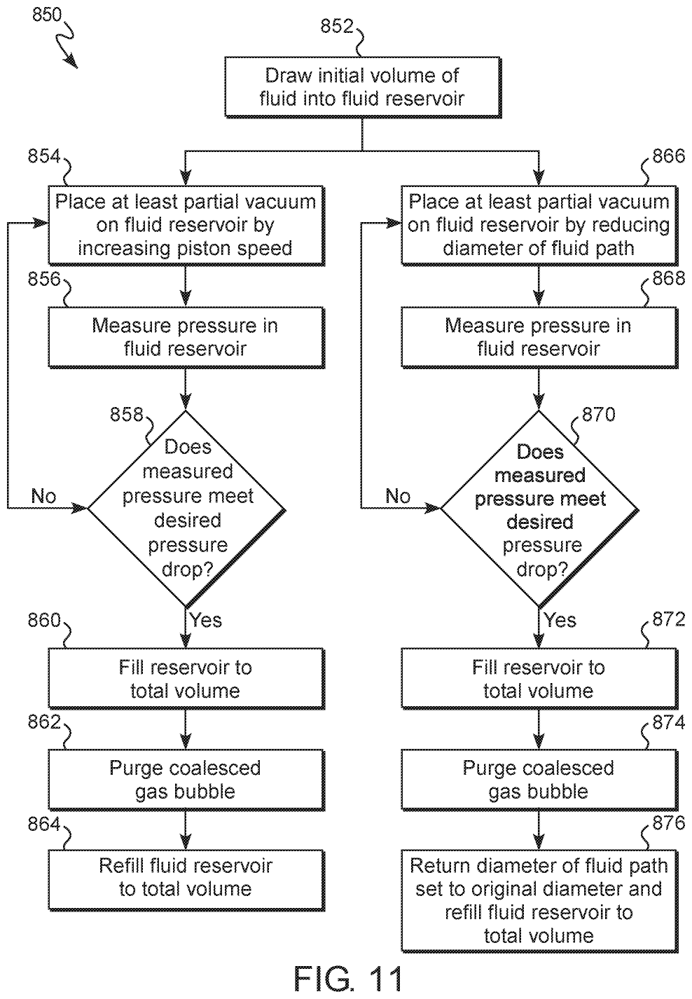

With reference now to FIG. 11-12B, at step 852 of the method 850, an initial volume of fluid is drawn into at least one fluid reservoir, e.g. at least one syringe 12, of the fluid injector system 100, similar to the manner in which fluid is drawn into the syringe 132 at step 802 of the method 800 for a closed or closeable system. Particularly, the initial volume of fluid may be drawn into the fluid reservoir by retracting the piston 103 (not shown in FIG. 5) connected to the plunger 14 in substantially the same manner described in step 802 of the method 800 for a closed or closeable system. The initial volume of fluid drawn into the fluid reservoir may be a portion of the desired final fill volume, for example, approximately 70% of a desired fill volume of the fluid reservoir. The desired fill volume may correspond to at least the prescribed volume of fluid to be injected into the patient according to a predetermined diagnostic injection protocol. In examples in which the fluid reservoir is the syringe 12, the location of the plunger 14 within the syringe 12, once the initial volume has been drawn into the syringe 12, may correspond to the starting position x₀.

With continued reference to FIG. 11-12B, at steps 854-858, an at least partial vacuum may be placed on the fluid reservoir, e.g. the at least one syringe 12. The at least partial vacuum may be created by generating a pressure drop across the open fluid injector system 100 by retracting the piston and plunger at a rate greater than the vacuum can be replaced. In a general form, the pressure drop may be calculated using equation (1):

$$Δ P = A ( \frac{η QL}{{d}^{4}} ) \qquad ( 1 )$$

wherein ΔP is the pressure drop across the open fluid injector system 100 (which may be measured in any appropriate pressure unit, such as in pounds per square inch (“psi”), atmospheres (atm), or Pascals (“Pa”)), η is a viscosity of at least one fluid being delivered from the fluid injector system to the patient (which may be measured in any appropriate viscosity unit, such as in centipoise (“CP”), A is a geometric constant of the fluid path, Q is the volumetric flow rate of at least one fluid being pulled into the fluid injector system through the outlet (which may be measured in any appropriate volumetric flow rate unit, such as in (“mL/s”)), L is the length of the fluid path, and d represents a diameter of the fluid path. Parameters A, L and d will vary depending upon the fluid path or the portion of the fluid injector system being measured. Yet, one skilled in the art would appreciate that other factors, such as fluid path geometry or orientation, may be able to be manipulated in order to obtain a desired pressure drop. The desired pressure drop ΔP represents the at least partial vacuum needed to overcome the atmospheric pressure A that the open fluid injector system is exposed to via the outlet 24, and to dislodge and coalesce any gas bubbles present in the initial volume of fluid drawn into the fluid reservoir.

As may be appreciated from the above pressure drop equation, the desired pressure drop ΔP for a given fluid may be obtained by altering either the flow rate Q, the diameter d at a portion of the fluid path, the length L of the fluid path, or a combination thereof. In one example of the present disclosure, described in steps 854-864, the pressure drop ΔP, and hence the at least partial vacuum, may be generated through an incremental change in flow rate Q of the at least one fluid. In particular, after the initial volume of fluid has been drawn into the fluid reservoir at step 852, or concurrently with drawing the initial volume of fluid into the fluid reservoir at step 852, the at least partial vacuum is placed on the fluid reservoir at step 854. In examples in which the fluid reservoir is the syringe 12, the at least partial vacuum may be created by the electronic control module 900 actuating the piston 103 to move the plunger 14 proximally from the starting position x₀ towards the final ending position x_{f}. The electronic control module 900 may first actuate the piston 103 at a predetermined initial speed, which may be later adjusted, if necessary, to achieve the desired pressure drop ΔP. In particular, at step 856, the actual pressure within the fluid reservoir may be intermittently or continuously monitored as the piston 103 moves the plunger 14 between the starting position x₀, and the final ending position x_{f}. Measurement of the pressure may be obtained, for example, via monitoring of the motor current of the motor driving the piston 103, monitoring the position of the plunger within the syringe, optical measurement of reservoir deformation, or by a pressure sensor in communication with the fluid path set 17 or the piston 103. At step 858, the electronic control module 900 determines whether the measured pressure drop meets or exceeds the desired pressure drop ΔP, and, consequently, the at least partial vacuum necessary to dislodge and coalesce the gas bubbles present in the initial volume of fluid drawn into fluid reservoir. The process by which the bubbles dislodge from at least one interior surface of the fluid reservoir (e.g. the sidewall of the syringe 12 and/or a surface of the plunger 14) and coalesce into a coalesced bubble in the fluid reservoir is substantially the same as the process described with reference to FIGS. 8A-8C.

Referring again to step 858, if the electronic control module 900 determines that the measured pressure in the fluid reservoir is below the desired pressure drop ΔP, the electronic control module 900 may return to step 854 and increase the speed of the piston 103. The pressure in the fluid reservoir is then re-measured at step 856 to determine if the pressure meets or exceeds the desired pressure drop ΔP. Steps 854 and 856 may be repeated as many times as necessary to achieve the desired pressure drop ΔP, with the speed of the piston 103 being incrementally increased at each iteration. According to various embodiments, repetition of steps 854 and 856 may result in additional impact forces to the syringe that may assist in dislodging the gas bubbles from the interior surfaces. In some examples, as shown in FIGS. 12A and 12B, the electronic control module 900 may be programmed to automatically increase the retraction speed of the piston 103. For example, electronic control module 900 may initially retract the piston 103 at a first speed V1 of 1 mL/s, as shown in FIG. 12A. The electronic control module 900 may then increase the retraction speed of the piston 103 to a second speed V2 of 2 mL/s, as shown in FIG. 12B, if the desired pressure drop ΔP is not detected at step 856. A first vacuum pressure P1 in the fluid reservoir generated by the first speed V1 may be less than a second vacuum pressure P2 generated by the second speed V2. The speed of retraction of the piston 103 may be continually increased in any predetermined or dynamically calculated increment until the desired pressure drop ΔP has been attained.

If at step 858, the electronic control module 900 determines that the measured pressure in the fluid reservoir meets or exceeds the desired pressure drop ΔP, the electronic control unit 900 may cease altering the speed of the piston 103. In some examples, if the measured pressure in fluid reservoir exceeds the desired pressure drop ΔP by a predetermined value, step 856 may be repeated except that the retraction speed of the piston 103 may be reduced to reduce the pressure drop.

With continued reference to FIG. 11, at step 860, the fluid reservoir may optionally be filled to the total volume prescribed by the diagnostic injection protocol. In some examples, the electronic control module 900 may actuate the piston 103 at a constant speed until the plunger 14 has reached the final ending position x_{f} within the syringe 12. The final ending position x_{f} may correspond to a total volume (i.e. 100% volume) of the fluid required by the diagnostic injection protocol. However, at this stage, some of the volume of the syringe 12 between the final ending position x_{f} and the outlet 24 may be occupied by the coalesced air bubble. In other examples, the final ending position x_{f} may correspond to a volume greater than the volume of fluid required by the diagnostic injection protocol, such that the total volume of fluid contained within the fluid reservoir after expulsion of the gas meets or exceeds the volume of fluid required by the diagnostic injection protocol.

In some examples, a flow control device, such as a flow diverter, may be positioned in proximity to the outlet 24. The flow diverter may direct the flow of fluid into the syringe 12 so as to avoid breaking or dividing the coalesced bubble formed in the syringe 12. In particular, the flow diverter may induce a Coand\check{a} effect such that incoming fluid flows around an outer surface of the coalesced bubble, thereby mitigating forces of fluid flow that could otherwise overcome the surface tension of the coalesced bubble and cause the coalesced bubble to fracture into multiple smaller bubbles.

With continued reference to FIG. 11, at step 862, the coalesced bubble is purged from the at least one fluid reservoir in the same manner described in step 814 of the method 800 of FIG. 7. At step 864, the at least one fluid reservoir is filled to the total volume (i.e. 100% volume) of fluid prescribed by the diagnostic injection protocol in the same manner described in step 816 of the method 800 of FIG. 7.

As noted above, steps 854-864 correspond to examples of the present disclosure in which the desired pressure drop ΔP is achieved by altering the flow rate Q of the fluid. In other examples, the desired pressure drop ΔP is achieved by altering the diameter d of at least a portion of the fluid path set 17. In such examples, steps 866-876 of the method 850 may be performed in place of steps 854-864.

In particular, after the initial volume of fluid has been drawn into the fluid reservoir at step 852, or concurrently with drawing the initial volume of fluid into the fluid reservoir at step 852, the diameter d of at least a portion of the fluid path set 17 may be decreased at step 866 to increase the pressure drop ΔP across the decreased diameter as the fluid is drawn through the fluid path set 17. In particular, the decreased diameter creates a restriction within the fluid path set 17 such that fluid exits the decreased diameter at a reduced pressure. In examples in which the fluid reservoir is the syringe 12, the electronic control module 900 may actuate the piston 103 to move the plunger 14 at a constant speed or at a varying speed (as described herein) from the starting position x₀ towards the final ending position x_{f}. As the fluid is drawn through the restriction created by the decreased diameter of the portion of the fluid path set 17, the pressure in the fluid reservoir decreases.

With continued reference to FIG. 11, at step 868, the actual pressure within the fluid reservoir may be intermittently or continuously monitored as the piston 103 moves the plunger 14 between the starting position x₀, and the final ending position x_{f}. Measurement of the pressure may be obtained via monitoring of the motor current of the motor driving the piston 103, monitoring the position of the plunger within the syringe 12, optical measurement of reservoir deformation, or by a pressure sensor in communication with the fluid path set 17 or the piston 103. At step 870, the electronic control module 900 determines whether the measured pressure drop meets or exceeds the desired pressure drop ΔP, and, consequently, the at least partial vacuum necessary to dislodge and coalesce the gas bubbles present in the initial volume of fluid drawn into fluid reservoir. The process by which the bubbles dislodge from at least one interior surface of the fluid reservoir (e.g. the sidewall of the syringe 12 and/or a surface of the plunger 14) and coalesce into a coalesced bubble in the fluid reservoir is substantially the same as the process described with reference to FIGS. 8A-8C.

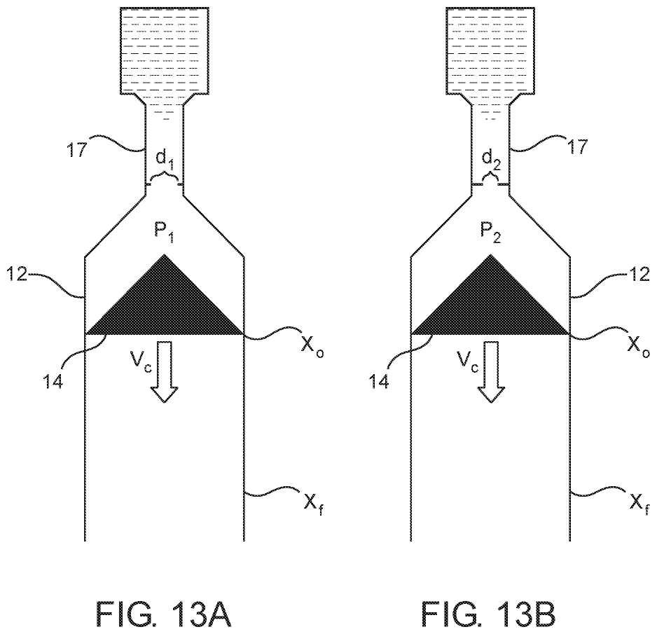

Referring again to step 870, if the electronic control module 900 determines that the measured pressure in the fluid reservoir is below the desired pressure drop ΔP, the electronic control module 900 may return to step 866 and further decrease the diameter d of the portion of the fluid path set 17, while the speed of the piston 103 remains constant. The pressure in the fluid reservoir is then re-measured at step 868 to determine if the pressure meets or exceeds the desired pressure drop ΔP. Steps 866 and 868 may be repeated as many times as necessary to achieve the desired pressure drop ΔP, with the diameter d of the portion of the fluid path set 17 being incrementally decreased at each iteration. In some examples, if the measured pressure in fluid reservoir exceeds the desired pressure drop ΔP by a predetermined value, step 866 may be repeated except that the diameter d may be increased to reduce the pressure drop. According to various embodiments, repetition of steps 866 and 868 may result in additional impact forces to the syringe that may assist in dislodging the gas bubbles from the interior surfaces. In some examples, as shown in FIGS. 13A and 13B, the electronic control module 900 may be programmed to automatically decrease the diameter d of the portion of the fluid path set 17.

In some examples, the electronic control module 900 may decrease the diameter d of the portion of the fluid path set 17 by closing a gate valve or pinch valve disposed in the fluid path set 17. In other examples, the electronic control module 900 may decrease the diameter d by modulating the opening and closing of a valve disposed in the fluid path set 17. In still other examples, the electronic control module 900 may decrease the diameter d by actuating a portion of the fluid path set 17 configured to change in size in response to temperature, voltage, current, magnetism, etc. The diameter may initially have a first diameter d1, as shown in FIG. 13A, while the plunger 14 is retracted at a constant velocity V_{c}. The electronic control module 900 may then decrease the diameter to a second diameter d2, as shown in FIG. 13B, if the desired pressure drop ΔP is not detected at step 856. The plunger 14 is maintained at the constant retraction velocity V_{c}. A first vacuum pressure P1 in the fluid reservoir resulting from the first diameter d1 may be less than a second vacuum pressure P2 resulting from the second diameter d2. The diameter of the portion of the fluid path set 17 may be continually decreased and/or increased in any desired increment until the desired pressure drop ΔP has been attained.

If at step 858, the electronic control module 900 determines that the measure pressure in the fluid reservoir meets or exceeds the desired pressure drop ΔP, the electronic control unit 900 may cease altering the diameter of the fluid path set 17.

With continued reference to FIG. 11, at step 872, the fluid reservoir may optionally be filled to the total volume prescribed by the diagnostic injection protocol in the same manner as step 860. At step 874, the coalesced bubble is purged from the at least one fluid reservoir in the same manner described in step 862. At step 876, the diameter of the portion of the fluid path set 17 is returned to its original diameter, and the at least one fluid reservoir is filled to the total volume (i.e. 100% volume) of fluid prescribed by the diagnostic injection protocol in the same manner described in step 864.

Having generally described the steps of the method 850, particular processes for adjusting the pressure drop ΔP in the fluid reservoir will now be described in greater detail. As described herein, the at least one fluid for filling the at least one fluid reservoir may be a saline solution, a contrast agent, or any other medical fluid that may be needed for a medical or diagnostic injection protocol. These fluids may have different viscosities, with more viscous fluids, such as the contrast agent, having characteristics such as concentration, more resistant to fluid flow than less viscous fluids, such as the saline solution. Further, different contrast agents may have different viscosities due to the concentrations of dissolved contrast and different saline flushing agents may similarly have different viscosities based on solution concentrations. Additionally, temperature of the fluid may have an effect on the viscosity of the fluid and the surface adhesion properties. As such, the fluid flow rate necessary to generate a given pressure drop may vary, at least in part, depending on the viscosity of the fluid. Additionally, the vacuum pressure necessary to cause the gas bubbles to dislodge and coalesce may vary, at least in part, based on the viscosity and other molecular and/or physical properties of the fluid. In particular, the molecular and/or physical properties of the given fluid may dictate, at least in part, the size increase of the bubbles necessary to overcome the surface adhesion of the bubbles to the at least one interior surface of the fluid reservoir. As such, at steps 854 and 866 of the method 850, the electronic control module 900 may determine or estimate the necessary vacuum pressure, i.e. the pressure drop ΔP, to overcome the surface adhesion of the bubbles based on known properties of the fluid and/or gas drawn into the fluid reservoir. For example, the electronic control module 900 may interpolate the necessary vacuum pressure from an empirical data set relating the initial and final bubble size to the necessary vacuum necessary to overcome the bubble adhesion force. In a similar manner, at step 866, the electronic control module 900 may estimate the necessary diameter of the portion of the fluid path set 17 based on known properties of the fluid and/or gas drawn into the fluid reservoir. In other examples, known properties of the fluid and/or gas may include at surface tension of the fluid relative to the interior surface, surface tension of the gas relative to the interior surface, surface texture of the at least one interior surface of the fluid reservoir, and buoyancy of the gas bubbles in the specific fluid.

In some examples of the present disclosure, the method 850, as performed by the fluid injector system 100, may be implemented by a computer program product. The computer program product may include at least one non-transitory computer-readable medium having one or more instructions executable by at least one processor to cause the at least one processor to execute all or part of the method 850. In some examples or aspects, the at least one non-transitory computer-readable medium and the at least one processor may include or correspond to the memory 908 and processor 904, respectively, as described above with reference to FIG. 4.

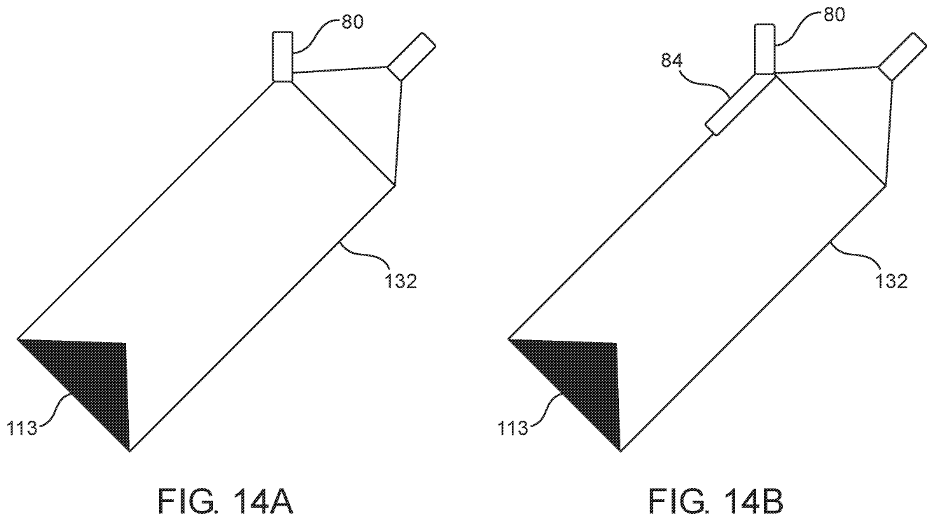

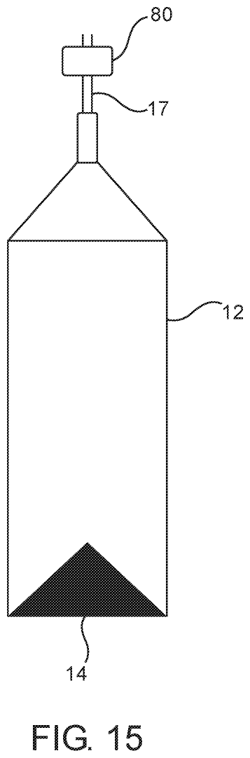

Referring now to FIGS. 14A-15, in some examples of the present disclosure, expulsion of the coalesced gas bubble 62 (see FIGS. 8A-8C) at step 814, 862, or 874 may be facilitated by at least one gas collection chamber 80 located within or attached to the fluid reservoir. Upon coalescing at the highest point of the fluid reservoir, e.g. the syringe 132, the at least one coalesced bubble 62 may accumulate in the gas collection chamber 80. In one example, illustrated in FIG. 14B, the gas collection chamber 80 may be in fluid communication with a channel 84 to guide the gas to the gas collection chamber 80. In another example, referring to FIG. 15, the gas collection chamber 80 may be located along at least one portion of the fluid path set 17 connected to the fluid reservoir, e.g. the syringe 12. The at least one coalesced air bubble may be expelled out of the fluid reservoir, e.g. the syringe 12, travel along the fluid path set 17, and accumulate within the gas collection chamber 80. Alternatively, the gas collection chamber 80 may be configured so that the coalesced bubble may be in a position where expulsion during a fluid injection protocol is not possible and the coalesced bubble is retained in the gas collection chamber 80. In still other embodiments, an adsorptive material may be located in the gas collection chamber 80 to react with or adsorb the gas, thereby removing the gas from the injection solution.

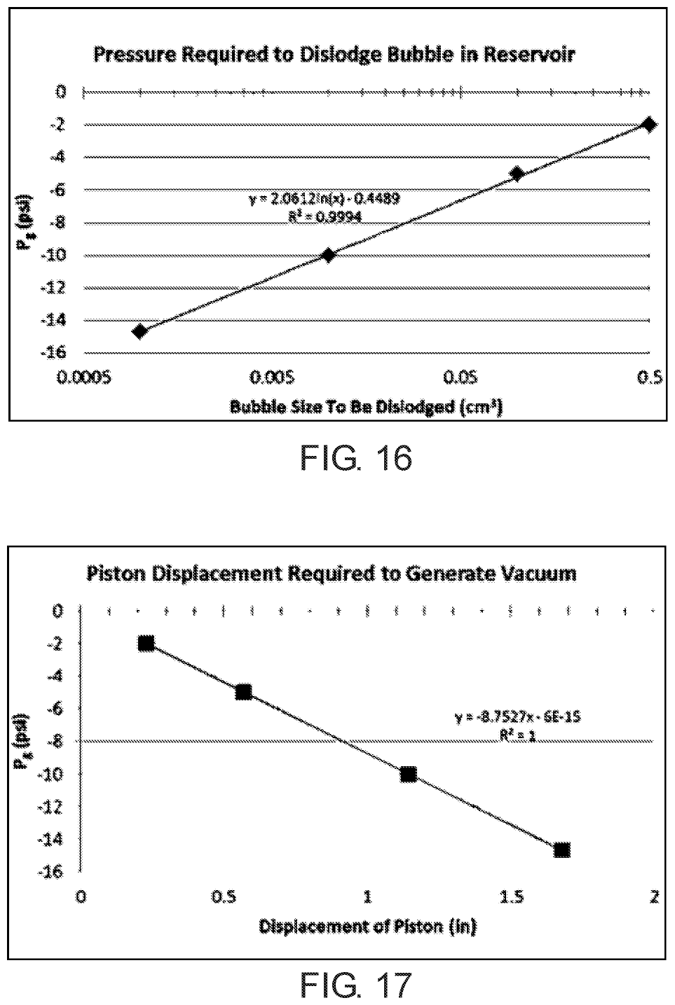

Referring now to FIGS. 16 and 17, in some examples of the methods 800 and 850, the at least partial vacuum generated at steps 806, 854, and 866 may be determined by implementation of an algorithm and/or a look-up table. In particular, the electronic control module 900 may be configured to determine the value of the at least partial vacuum for a given fluid based on an algorithm and/or a look-up table relating the size of the bubble to be dislodged to a necessary vacuum pressure. FIG. 16 shows a graphical representation of an equation relating bubble size in cubic centimeters (cm³) to necessary gauge pressure (P_{g}) for a given fluid contained within the fluid reservoir. Bubble size (cm³) increases along the x axis, while gauge pressure (P_{g}) decreases going down the y-axis. The electronic control module 900 may execute an algorithm incorporating the equation to determine the gauge pressure necessary to dislodge the bubbles from the at least one interior surface of the fluid reservoir. In some examples, the algorithm executed by the electronic control module 900 may further include an equation and/or a look-up table for determine the retraction of the piston 103 necessary to attain the determined vacuum. FIG. 17 shows a graphical representation of an equation relating displacement or retraction of the piston 103 in inches (in) to gauge pressure (P_{g}) for a given fluid reservoir. Piston displacement or retraction (in) increases along the x axis, while gauge pressure (P_{g}) decreases going down the y-axis. The equation for determining displacement of the piston 103 to achieve the necessary gauge pressure may be a function of the physical properties of the fluid reservoir, such as an internal cross sectional area of the fluid reservoir perpendicular to the retraction direction of the piston 103.

While several examples of fluid delivery systems, computer program products, and methods of use thereof are shown in the accompanying drawings and described hereinabove in detail, other examples will be apparent to, and readily made by, those skilled in the art without departing from the scope and spirit of the disclosure. For example, it is to be understood that this disclosure contemplates that, to the extent possible, one or more features of any example can be combined with one or more features of any other example. Accordingly, the foregoing description is intended to be illustrative rather than restrictive.

1. A fluid injector system, comprising:
at least one actuator configured to change the internal volume of the at least one fluid reservoir;
at least one fluid reservoir having at least one interior surface and defining an internal volume; and
at least one processor programmed or configured to:
drive the actuator to at least partially fill the at least one fluid reservoir with a fluid from a fluid source;
drive the actuator to generate an at least a partial vacuum within the internal volume to dislodge one or more gas bubbles adhered to the at least one interior surface and to cause the one or more gas bubbles to coalesce into a coalesced bubble; and
drive the actuator to expel the coalesced bubble from an outlet of the at least one fluid reservoir.

2. The fluid injector system of claim 1, wherein the at least one processor is further programmed or configured to, prior to driving the actuator to generate at least the partial vacuum within the internal volume, close the outlet of the at least one fluid reservoir to fluidly isolate the internal volume.

3. The fluid injector system of claim 2, wherein the at least one processor is further programmed or configured to, after closing the outlet of the at least one fluid reservoir, drive the actuator to pressurize the coalesced bubble.

4. The fluid injector system of claim 2, wherein the at least one processor is further programmed or configured to, prior to driving the actuator to expel the coalesced bubble from an outlet of the at least one fluid reservoir, open the outlet of the at least one fluid reservoir to provide fluid communication between the internal volume and one of the fluid source and a fluid outlet pathway.

5. (canceled)

6. (canceled)

7. (canceled)

8. (canceled)

9. The fluid injector system of claim 1, wherein the at least one processor is further programmed or configured to vibrate or oscillate at least one of the at least one interior surfaces to dislodge the one or more gas bubbles.

10. The fluid injector system of claim 2, wherein the at least one fluid reservoir further comprises a valve in fluid communication with the outlet of the at least one fluid reservoir, wherein the valve has at least a first open position and a second closed position, and wherein closing the outlet of the at least one fluid reservoir comprises moving the valve to the second closed position.

11. The fluid injector system of claim 10, wherein there is fluid communication between the internal volume of the at least one fluid reservoir and the fluid source when the valve is in the first open position, and wherein the valve further comprises a third open position allowing fluid communication between the internal volume of the at least one fluid reservoir and a fluid outlet pathway.

12. (canceled)

13. (canceled)

14. The fluid injector system of claim 1, wherein the at least one processor is further programmed or configured to drive the actuator to prime a fluid path set in fluid communication with a fluid outlet pathway after the coalesced bubble is expelled from the at least one fluid reservoir.

15. (canceled)

16. (canceled)

17. (canceled)

18. A method for removing gas bubbles from at least one fluid reservoir of a fluid injector system, the method comprising:
driving an actuator to at least partially fill the at least one fluid reservoir with a fluid from a fluid source;
driving the actuator to generate an at least partial vacuum within an internal volume of the at least one fluid reservoir to dislodge one or more gas bubbles adhered to least one interior surface of the at least one fluid reservoir and to cause the one or more gas bubbles to coalesce into a coalesced bubble; and
driving the actuator to expel the coalesced bubble from an outlet of the at least one fluid reservoir.

19. The method of claim 18, further comprising prior to driving the actuator to generate at least the partial vacuum within the internal volume, closing the outlet of the at least one fluid reservoir to fluidly isolate the internal volume.

20. The method of claim 19, further comprising after closing the outlet of the at least one fluid reservoir, driving the actuator to pressurize the coalesced bubble.

21. The method of claim 19, further comprising prior to driving the actuator to expel the coalesced bubble from an outlet of the at least one fluid reservoir, opening the outlet of the at least one fluid reservoir to provide fluid communication between the internal volume and one of the fluid source and a fluid outlet pathway.

22. (canceled)

23. (canceled)

24. The method of claim 18, further comprising vibrating at least one of the at least one interior surfaces to dislodge the one or more gas bubbles.

25. The method of claim 18, wherein closing the outlet of the at least one fluid reservoir comprises moving a valve at the outlet from a first open position where the internal volume is in fluid communication with the fluid source to a second closed position where the internal volume is fluidly isolated from the fluid source.

26. The method of claim 25, wherein there is fluid communication between the internal volume of the at least one fluid reservoir and the fluid source when the valve is in the first open position, and wherein the valve further comprises a third open position allowing fluid communication between the internal volume of the at least one fluid reservoir and a fluid outlet pathway.

27. The method of claim 18, further comprising driving the actuator to prime a fluid path set in fluid communication with reservoir fluid outlet pathway after the coalesced bubble is expelled from the at least one fluid reservoir.

28. The method of claim 18, further comprising determining the amount of vacuum necessary to dislodge the one or more gas bubbles from the at least one interior surface based one at least one of a surface tension of the fluid, a surface tension of the gas, a surface texture of the at least one interior surface, and buoyancy of the one or more gas bubble in the fluid.

29. The method of claim 18, further comprising:
measuring the pressure within the internal volume of the fluid reservoir; and
adjusting the at least partial vacuum within the internal volume based on the measured pressure.

30. The method of claim 29, wherein adjusting the at least partial vacuum comprises at least one of:
increasing or decreasing a speed of retraction of the actuator; and
increasing or reducing a diameter of at least a portion of a fluid path set in fluid communication with the internal volume of the fluid reservoir.

31. A method for removing gas bubbles from at least one fluid filled fluid reservoir of a fluid injector system, the method comprising:
generating at least a partial vacuum within an internal volume of the at least one fluid reservoir
dislodging one or more gas bubbles adhered to least one interior surface of the at least one fluid reservoir, wherein the vacuum causes the one or more dislodged gas bubbles to enlarge and coalesce into a coalesced bubble; and
expelling the coalesced bubble from an outlet of the at least one fluid reservoir.

32.-56. (canceled)
